(19) United States
(12) Patent Application Publication
(10) Pub. No.: US 2025/0135049 A1
(43) Pub. Date: May 1, 2025

(54) Methods for Biological Material Labeling and Medical Imaging

(71) Applicant: Mayo Foundation for Medical Education and Research, Rochester, MN (US)

(72) Inventors: Aditya Bansal, Rochester, MN (US); Timothy R. DeGrado, Arvada, CO (US); Mukesh K. Pandey, Rochester, MN (US)

(21) Appl. No.: 18/681,629

(22) PCT Filed: Aug. 5, 2022
(86) PCT No.: PCT/US2022/039582

§ 371 Date: Feb. 6, 2024

Related U.S. Application Data
(60) Provisional application No. 63/230,318, filed on Aug. 6, 2021.

(51) Int. Cl. A61K 51/12; A61K 51/04; A61K 51/10; C07B 59/00

(52) U.S. Cl. CPC A61K 51/1203; A61K 51/0497; A61K 51/1093; C07B 59/008; A61K 2123/00; C07B 2200/05

(57) ABSTRACT

A method for preparing a labeling agent includes: providing a compound including a chelating moiety and a conjugation moiety; contacting the compound with a radionuclide to create a radiolabeled preparation having a first molar activity; and purifying the radiolabeled preparation to prepare a labeling agent having a second molar activity that is greater than the first molar activity. A biological material can be contacted with a labeling agent prepared by the method such that the biological material becomes labeled for imaging. In one example embodiment, there is provided a way of synthesizing a labeling agent, such as ⁸⁹Zr-labeled p-isothiocyana to-benzyl-desferrioxamine, using a purification step that results in increased molar activity and labeling efficiency, which makes possible the successful labeling of biological materials at very low concentrations where an unpurified labeling agent would be unsuccessful.

### CROSS-REFERENCES TO RELATED APPLICATIONS

This application is based on, claims benefit of, and claims priority to U.S. Application No. 63/230,318 filed on Aug. 6, 2021, which is hereby incorporated by reference herein in its entirety for all purposes.

### STATEMENT REGARDING FEDERALLY SPONSORED RESEARCH

Not Applicable.

### BACKGROUND OF THE INVENTION

### 1. Field of the Invention

This invention relates to methods of labeling of a biological material for medical imaging, methods for preparing a labeling agent, and methods for medical imaging of a subject using a biological material labeled with a labeling agent.

### 2. Description of the Related Art

In cell therapy, cells are injected into the body as a therapy against diseases such as some cancers, or to regenerate damaged tissues. This type of treatment benefits from the ability to image cells as they migrate though the body in order to evaluate therapy progress. A number of radioisotopic cell labeling methods have traditionally been used for single-photon emission computerized tomography (SPECT) and positron emission tomography (PET) imaging-based cell tracking (see Nguyen et al., [Ref. 81]) (The full citation for all numbered references (i.e., ending with Ref. XX) mentioned throughout the specification can be found in the “References” section of the specification (i.e., ¶ [00149]). As described below, the citation of any document or reference is not to be construed as an admission that it is prior art with respect to the present invention.) However, a PET-based approach offers superior quantification and imaging sensitivity characteristics over a SPECT-based approach, which are critical for tracking of small numbers of administered cells (see Nguyen et al., [Ref. 81]). In this regard, ⁸⁹Zr has emerged as an attractive PET radionuclide for cell labeling applications due to its high spatial resolution and 78.4-hour half-life that may allow monitoring of administered cells up to a 2-3 week period. U.S. Patent Application Publication No. 2018/0043041 to Aditya Bansal et al. discloses methods of ex vivo labeling of a biological material for in vivo imaging, methods of labeling a biological material in vivo, methods for preparing a labeling agent, and methods for in vivo imaging of a subject using a biological material labeled with a labeling agent. With respect to labeling agents, U.S. 2018/0043041 discloses a method of synthesizing ⁸⁹Zr-labeled p-isothiocyanato-benzyl-desferrioxamine (⁸⁹Zr-DBN), a labeling agent that uses ⁸⁹Zr as a long-half-life radionuclide (3.3 days), enabling PET imaging of labeled injected biological material for several weeks. The ⁸⁹Zr-DBN can act as a general Zr-89 labeling synthon and can be produced at a centralized facility and shipped to various sites and labs that need to perform labeling of biological materials such as, without limitation, cells, liposomes, DNA aptamers, RNA aptamers, viruses, nanoparticles, extracellular vesicles, microorganisms, antibodies, proteins, peptides, scaffolds, polymers, and nucleic acids.

Even with the technical advances provided by the methods and labeling agents of U.S. 2018/0043041, the labeling of biological materials (such as some viruses) at very low concentrations can be difficult and/or unsuccessful. Therefore, there is a need for even more improved labeling agents which make possible the successful labeling of biological materials at very low concentrations.

### SUMMARY OF THE INVENTION

The present disclosure provides a new way of synthesizing a labeling agent, such as ⁸⁹Zr-labeled p-isothiocyanato-benzyl-desferrioxamine (⁸⁹Zr-DBN), using a purification step that results in 2-3-fold increased molar activity and labeling efficiency, which makes possible the successful labeling of biological materials (such as some viruses or extracellular vesicles) at very low concentrations where an unpurified labeling agent would be difficult to radiolabel and can thus result in poor labeling.

In one aspect, the present disclosure provides a method for preparing a labeling agent. The method comprises: (a) providing a compound including a chelating moiety and a conjugation moiety; (b) contacting the compound with a radionuclide to create a radiolabeled preparation having a first molar activity measured at an end of step (b); and (c) purifying the radiolabeled preparation to prepare a labeling agent having a second molar activity measured at an end of step (c), wherein the second molar activity is greater than the first molar activity. The second molar activity can be at least two times greater than the first molar activity.

In some aspects, a second molar activity can be at least two times greater than a first molar activity.

In some aspects, a first molar activity can be in a range of 1 to 50 GBq/μmol.

In some aspects, a second molar activity can be in a range of 100 to 500 GBq/μmol.

In some aspects, a radionuclide can be selected from the group consisting of ¹¹C, ¹³N, ¹⁵O, ¹⁸F, ^{34m}Cl, ³⁸K, ⁴⁵Ti, ⁵¹Mn, ⁵²Mn, ^{52m}Mn, ⁵²Fe, ⁵⁵Co, ⁶⁰Cu, ⁶¹Cu, ⁶²Cu, ⁶⁴Cu, ⁶⁶Ga, ⁶⁸Ga, ⁷¹As, ⁷²As, ⁷⁴As, ⁷⁵Br, ⁷⁶Br, ⁸²Rb, ⁸⁶Y, ⁸⁹Zr, ⁹⁰Nb, ^{94m}Tc, ^{99m}Tc, ^{110m}In, ¹¹¹In, ¹¹⁸Sb, ¹²⁰I, ²⁰³Pb: ¹²¹I, ¹²²I, ¹²³I, and ¹²⁴I.

In some aspects, step (b) can include contacting a compound with a solution of a halide including a radionuclide cation.

In some aspects, a radionuclide cation can be ⁸⁹Zr⁺⁴.

In some aspects, a halide can be chloride (Cl⁻).

In some aspects, step (b) can include contacting a compound with ⁸⁹Zr-chloride in a hydrochloride solution.

In some aspects, step (b) can include contacting a compound with ⁸⁹Zr-chloride in a hydrochloride solution at a pH in a range of 7 to 9.

In some aspects, step (c) can include purifying a radiolabeled preparation using reverse phase chromatography.

In some aspects, step (c) can include purifying a radiolabeled preparation using gradient elution.

In some aspects, gradient elution can use at least two different solvents.

In some aspects, one of the solvents can include water and trifluoroacetic acid, and another of the solvents can include acetonitrile and trifluoroacetic acid.

In some aspects, a chelating moiety can be a hydroxamic acid group.

In some aspects, a hydroxamic acid group can be a desferrioxamine group.

In some aspects, a conjugation moiety can include an isothiocyanate group.

In some aspects, a conjugation moiety can include a benzyl group.

In some aspects, a labeling agent can be a ⁸⁹Zr-isothiocyanato-benzyl-desferrioxamine.

In some aspects, a labeling agent can have a radiochemical stability greater than 60% measured at 72 hours after step (c).

In some aspects, a method can include (d) adding a stabilizer to a labeling agent.

In some aspects, a stabilizer can be ascorbic acid.

In some aspects, a labeling agent can have a radiochemical stability greater than 80% measured at 72 hours after step (d).

In some aspects, step (b) can include creating a radiolabeled preparation at a radiochemical yield of at least 95%.

In another aspect, the present disclosure provides a method of labeling of a biological material for imaging. The method comprises contacting a biological material with the labeling agent prepared by the method of this disclosure such that the biological material becomes labeled for imaging, wherein the biological material is selected from cells, liposomes, DNA aptamers, RNA aptamers, viruses, nanoparticles, extracellular vesicles, microorganisms, antibodies, proteins, peptides, scaffolds, polymers, and nucleic acids.

In some aspects, a radiolabeling yield when contacting a biological material with a labeling agent can be at least 5% when the biological material is contacted with the labeling agent at a concentration of the biological material of 0.1 mg/mL.

In some aspects, a radiolabeling yield when contacting a biological material with a labeling agent can be at least 15% when the biological material is contacted with the labeling agent at a concentration of the biological material of 0.5 mg/mL.

In some aspects, a radiolabeling yield when contacting a biological material with a labeling agent can be at least 30% when the biological material is contacted with the labeling agent at a concentration of the biological material of 1.0 mg/mL.

In some aspects, a first radiolabeling yield when contacting a first amount of a biological material with a first quantity of a labeling agent can be greater than a second radiolabeling yield when contacting a second amount of the biological material with a second quantity of a radiolabeled preparation created in step (b). The first amount and the second amount can be the same. The first quantity and the second quantity can be the same.

In some aspects, a first radiolabeling yield when contacting a first amount of a biological material with a first quantity of a labeling agent can be at least two times greater than a second radiolabeling yield when contacting a second amount of the biological material with a second quantity of a radiolabeled preparation created in step (b). The first amount and the second amount can be the same. The first quantity and the second quantity can be the same.

In some aspects, a biological material can be selected from antibodies. The biological material can include an antibody.

In some aspects, a biological material can be selected from proteins. The biological material can include a protein.

In some aspects, a biological material can be selected from cells. The biological material can include a cell.

In some aspects, a biological material can be selected from viruses. The biological material can include a virus.

In some aspects, a biological material can be selected from stem cells. The biological material can include a stem cell.

In some aspects, a biological material can be selected from white blood cells. The biological material can include a white blood cell.

In another aspect, the present disclosure provides a method for in vivo imaging of a subject. The method can include (a) administering to the subject a biological material labeled with a labeling agent prepared by a method of this disclosure, (b) waiting a time sufficient to allow the biological material to accumulate at a tissue site to be imaged, and (c) imaging the tissues with a non-invasive imaging technique. The biological material can be selected from cells, liposomes, DNA aptamers, RNA aptamers, viruses, nanoparticles, microorganisms, antibodies, proteins, peptides, scaffolds, polymers, and nucleic acids.

In some aspects, a non-invasive imaging technique can be selected from positron emission tomography imaging, positron emission tomography with computed tomography imaging, or positron emission tomography with magnetic resonance imaging.

In another aspect, the present disclosure provides a method of imaging a subject by emission tomography. The method can include (a) administering to the subject a biological material labeled with a labeling agent prepared by a method of the present disclosure, (b) using a plurality of detectors to detect gamma rays emitted from the subject and to communicate signals corresponding to the detected gamma rays, and (c) reconstructing from the signals a series of medical images of a region of interest of the subject. The biological material can be selected from cells, liposomes, DNA aptamers, RNA aptamers, viruses, nanoparticles, microorganisms, antibodies, proteins, peptides, scaffolds, polymers, and nucleic acids.

In another aspect, the preset disclosure provides an imaging method. The imaging method can include acquiring an image of a subject to whom a detectable amount of a biological material labeled with a labeling agent prepared by a method of the presented disclosure has been administered. The biological material can be selected from cells, liposomes, DNA aptamers, RNA aptamers, viruses, nanoparticles, microorganisms, antibodies, proteins, peptides, scaffolds, polymers, and nucleic acids.

In some aspects, a method can include acquiring an image using positron emission tomography imaging, positron emission tomography with computed tomography imaging, or positron emission tomography with magnetic resonance imaging.

In some aspects, a biological material can be selected from antibodies.

In some aspects, a biological material can be selected from proteins.

In some aspects, a biological material can be selected from cells.

In some aspects, a biological material can be selected from viruses.

In some aspects, a biological material can be selected from stem cells.

In some aspects, a biological material can be selected from white blood cells.

In yet another aspect, the present disclosure provides a method for determining radiolabeling efficiency when a biological material is contacted with a labeling agent including a radionuclide to produce a radiolabeled biological material. The method comprises separating the radiolabeled biological material produced when the biological material is contacted with the labeling agent from free radionuclide and unconjugated labeling agent using instant thin layer chromatography.

In some aspects, a labeling agent can be the labeling agent prepared by a method of this disclosure.

In some aspects, a biological material can be selected from cells, liposomes, DNA aptamers, RNA aptamers, viruses, nanoparticles, microorganisms, antibodies, proteins, peptides, scaffolds, polymers, and nucleic acids.

In some aspects, a biological material can be selected from antibodies.

In some aspects, instant thin layer chromatography can use an acid-alcohol mixture as a mobile phase and a gel as a solid phase.

In some aspects, an acid-alcohol mixture can include citric acid and methanol.

In some aspects, a gel can include silica.

These and other features, aspects, and advantages of the present invention will become better understood upon consideration of the following detailed description, drawings and appended claims.

### BRIEF DESCRIPTION OF THE DRAWINGS

FIG. 1 is a schematic of a positron emission tomography (PET) system.

FIG. 2 shows a comparison of HPLC traces of unpurified and purified [⁸⁹Zr] Zr-DBN.

FIG. 3 shows a comparison of radiolabeling yield of antibody (IgG) with purified and unpurified [⁸⁹Zr] Zr-DBN as a function of protein (IgG) concentration.

FIG. 4 shows a comparison of HPLC trace and peak analysis of purified [⁸⁹Zr] Zr-DBN with and without addition of ascorbic acid.

FIG. 5 shows a comparison of HPLC trace and peak analysis of purified [⁸⁹Zr] Zr-DBN with and without addition of ascorbic acid at 72 hours post HPLC purification.

FIG. 6 shows an rTLC analysis of [⁸⁹Zr] Zr-DBN in 100 mM DTPA (pH 7) solution.

FIG. 7 shows a concentration calibration curve for DFO-NCS.

FIG. 8 shows an rTLC analysis of [⁸⁹Zr] Zr-chloride in 20 mM citric acid (pH 4.9-5.1) 1:1 methanol (v:v) solution.

FIG. 9 shows an rTLC analysis of [⁸⁹Zr] Zr-DBN in 20 mM citric acid (pH 4.9-5.1) 1:1 methanol (v:v) solution.

FIG. 10 shows an rTLC analysis of [⁸⁹Zr] Zr-IgG and [⁸⁹Zr] Zr-DBN in 20 mM citric acid (pH 4.9-5.1) 1:1 methanol (v:v) solution.

FIG. 11 shows in panels A-C:HPGe spectrums of ⁸⁹Zr produced at timepoint ⁸⁸Zr and ⁸⁸Y. Panel A: HPGe spectrum of ⁸⁹Zr. Panel B: HPGe spectrum of ⁸⁹Zr 60 days after the EOB. Panel C:HPGe spectrum of ⁸⁹Zr 96 days after the EOB (produced at 15.2 MeV beam energy).

FIG. 12 shows an HPLC trace of [⁸⁹Zr] Zr-chloride.

FIG. 13 shows chemical structures of [⁸⁹Zr] Zr-DFO-Bn-NCS.

FIG. 14 shows percentage of ⁸⁹Zr complexation with DFO-Bn-NCS, at different reaction times.

FIG. 15 shows radiolabeling of white blood cells (WBCs) with [⁸⁹Zr] Zr-DFO-Bn-NCS.

FIG. 16 shows radiolabeling of stem cells with [⁸⁹Zr] Zr-DFO-Bn-NCS.

FIG. 17 shows representative PET images showing distribution of WBCs labeled with [⁸⁹Zr] Zr-DFO-NCS in athymic nude mice at different time points post-injection.

FIG. 18 shows standardized uptake value (SUV) and biodistribution of WBCs labeled with [⁸⁹Zr] Zr-DFO-NCS in major organs of athymic nude mice at day 7 post-injection . . .

FIG. 19 shows standardized uptake value (SUV) and biodistribution of WBCs labeled with [⁸⁹Zr] Zr-DFO-NCS in athymic nude mice at day 7 post-injection.

FIG. 20 shows representative PET images showing distribution of stem cells labeled with [⁸⁹Zr] Zr-DFO-NCS in athymic nude mice at different time points post-injection.

FIG. 21 shows standardized uptake value (SUV) and biodistribution of stem cells labeled with [⁸⁹Zr] Zr-DFO-NCS in lung, liver and spleen of athymic nude mice at day 7 post-injection.

FIG. 22 shows standardized uptake value (SUV) and biodistribution of stem cells labeled with [⁸⁹Zr] Zr-DFO-NCS, in lung, liver and spleen of athymic nude mice at day 7 post-injection.

FIG. 23 shows representative PET images showing distribution of [⁸⁹Zr] ZrCl₄ in athymic nude mice at different time points post-injection.

FIG. 24 shows standardized uptake value (SUV) and distribution of [⁸⁹Zr] ZrCl₄ in athymic nude mice at day 7 post-injection.

### DETAILED DESCRIPTION OF THE INVENTION

The present disclosure provides a new way of synthesizing a labeling agent, such as ⁸⁹Zr-DBN, using a purification step that results in 2-3 fold increased molar activity and labeling efficiency, which makes possible the successful labeling of biological materials (such as some viruses) at very low concentrations where an unpurified labeling agent would be difficult to radiolabel and can thus result in poor labeling.

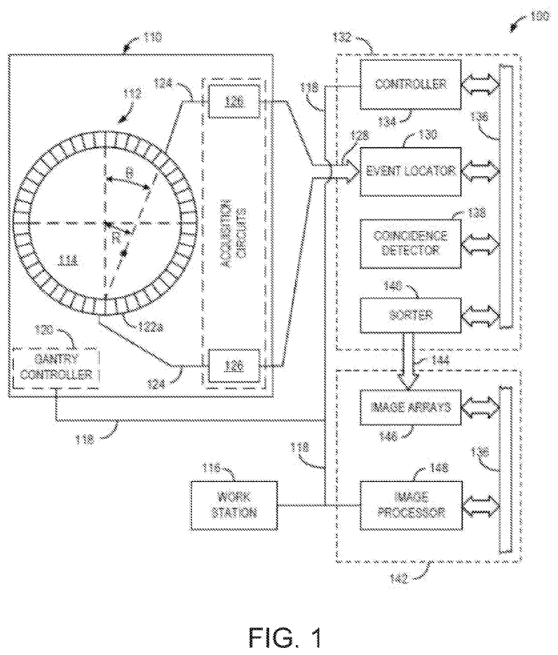

Referring now to FIG. 1, a PET system 100 that can be used with the labeled biological material of the present invention comprises an imaging hardware system 110 that includes a detector ring assembly 112 about a central axis or bore 114. An operator workstation 116 including a commercially available processor running a commercially available operating system communicates through a communications link 118 with a gantry controller 120 to control operation of the imaging hardware system 110.

The detector ring assembly 112 is formed of a multitude of radiation detector units 122 that produce a signal responsive to detection of a photon on communications line 124 when an event occurs. A set of acquisition circuits 126 receive the signals and produce signals indicating the event coordinates (x, y) and the total energy associated with the photons that caused the event. These signals are sent through a cable 128 to an event locator circuit 130. Each acquisition circuit 126 also produces an event detection pulse that indicates the exact moment the interaction took place. Other systems utilize sophisticated digital electronics that can also obtain this information regarding the precise instant in which the event occurred from the same signals used to obtain energy and event coordinates.

The event locator circuits 130 in some implementations, form part of a data acquisition processing system 132 that periodically samples the signals produced by the acquisition circuits 126. The data acquisition processing system 132 includes a general controller 134 that controls communications on a backplane bus 136 and on the general communications network 118. The event locator circuits 130 assemble the information regarding each valid event into a set of numbers that indicate precisely when the event took place and the position in which the event was detected. This event data packet is conveyed to a coincidence detector 138 that is also part of the data acquisition processing system 132.

The coincidence detector 138 accepts the event data packets from the event locator circuit 130 and determines if any two of them are in coincidence. Coincidence is determined by a number of factors. First, the time markers in each event data packet must be within a predetermined time window, for example, 0.5 nanoseconds or even down to picoseconds. Second, the locations indicated by the two event data packets must lie on a straight line that passes through the field of view in the scanner bore 114. Events that cannot be paired are discarded from consideration by the coincidence detector 138, but coincident event pairs are located and recorded as a coincidence data packet. These coincidence data packets are provided to a sorter 140. The function of the sorter in many traditional PET imaging systems is to receive the coincidence data packets and generate memory addresses from the coincidence data packets for the efficient storage of the coincidence data. In that context, the set of all projection rays that point in the same direction (θ) and pass through the scanner's field of view (FOV) is a complete projection, or “view”. The distance (R) between a particular projection ray and the center of the FOV locates that projection ray within the FOV. The sorter 140 counts all of the events that occur on a given projection ray (R, θ) during the scan by sorting out the coincidence data packets that indicate an event at the two detectors lying on this projection ray. The coincidence counts are organized, for example, as a set of two-dimensional arrays, one for each axial image plane, and each having as one of its dimensions the projection angle θ and the other dimension the distance R. This θ by R map of the measured events is call a histogram or, more commonly, a sinogram array. It is these sinograms that are processed to reconstruct images that indicate the number of events that took place at each image pixel location during the scan. The sorter 140 counts all events occurring along each projection ray (R, θ) and organizes them into an image data array.

The sorter 140 provides image datasets to an image processing/reconstruction system 142, for example, by way of a communications link 144 to be stored in an image array 146. The image arrays 146 hold the respective datasets for access by an image processor 148 that reconstructs images. The image processing/reconstruction system 142 may communicate with and/or be integrated with the work station 116 or other remote work stations.

The PET system 100 provides an example emission tomography system for acquiring a series of medical images of a subject during an imaging process after administering a pharmaceutically acceptable composition including labeled biological materials as described herein. The system includes a plurality of detectors configured to be arranged about the subject to acquire gamma rays emitted from the subject over a time period relative to an administration of the composition to the subject and communicate signals corresponding to acquired gamma rays. The system also includes a reconstruction system configured to receive the signals and reconstruct therefrom a series of medical images of the subject. In one version of the system, a second series of medical images is concurrently acquired using an x-ray computed tomography imaging device. In one version of the system, a second series of medical images is concurrently acquired using a magnetic resonance imaging device.

Administration to the subject of a pharmaceutical composition including radiolabeled biological materials for in vivo detection of the accumulated biological materials in a target region of the subject can be accomplished intravenously, intraarterially, intrathecally, intramuscularly, intradermally, subcutaneously, or intracavitary. A “subject” is a mammal, preferably a human. In the method of the invention, sufficient time is allowed after administration of a detectable amount of the radiolabeled biological materials such that the radiolabeled biological materials can accumulate in a target region of the subject. A “detectable amount” means that the amount of the detectable radiolabeled biological materials that is administered is sufficient to enable detection of accumulation of the radiolabeled biological materials in a subject by a medical imaging technique.

One non-limiting example method of imaging according to the invention involves the use of an intravenous injectable composition including radiolabeled biological materials. A positron emitting atom of the radiolabeled biological materials gives off a positron, which subsequently annihilates and gives off coincident gamma radiation. This high energy gamma radiation is detectable outside the body using positron emission tomography imaging, or positron emission tomography concurrent with computed tomography imaging (PET/CT). With PET/CT, the location of the injected and subsequently accumulated labeled biological materials within the body can be identified.

In one embodiment, the present invention provides a method for preparing a labeling agent. The method comprises: (a) providing a compound including a chelating moiety and a conjugation moiety; (b) contacting the compound with a radionuclide to create a radiolabeled preparation having a first molar activity measured at an end of step (b); and (c) purifying the radiolabeled preparation to prepare a labeling agent having a second molar activity measured at an end of step (c), wherein the second molar activity is greater than the first molar activity. The second molar activity can be at least two times greater than the first molar activity. The second molar activity can be at least two times greater than the first molar activity. The first molar activity can be in a range of 1 to 50 GBq/μmol. The second molar activity can be in a range of 100 to 500 GBq/μmol.

In the method, the chelating moiety can be a hydroxamic acid group. The hydroxamic acid group can be a desferrioxamine group. The conjugation moiety can include an isothiocyanate group. The conjugation moiety can include a benzyl group. The labeling agent can be ⁸⁹Zr-isothiocyanato-benzyl-desferrioxamine.

The radionuclide can selected from the group consisting of ¹¹C, ¹³N, ¹⁵O, ¹⁸F, ^{34m}Cl, ³⁸K, ⁴⁵Ti, ⁵¹Mn, ⁵²Mn, ^{52m}Mn, ⁵²Fe, ⁵⁵Co, ⁶⁰Cu, ⁶¹Cu, ⁶²Cu, ⁶⁴Cu, ⁶⁶Ga, ⁶⁸Ga, ⁷¹As, ⁷²As, ⁷⁴As, ⁷⁵Br, ⁷⁶Br, ⁸²Rb, ⁸⁶Y, ⁸⁹Zr, ⁹⁰Nb, ^{94m}Tc, ^{99m}Tc, ^{110m}In, ¹¹¹In, ¹¹⁸Sb, ¹²⁰I, ¹²¹I, ¹²²I, ¹²³I, and ¹²⁴I. Step (b) of the method can comprise contacting the compound with a solution of a halide including a radionuclide cation. The radionuclide cation can be ⁸⁹Zr⁺⁴. The halide can be chloride (CI). Step (b) of the method can comprise contacting the compound with ⁸⁹Zr-chloride in a hydrochloride solution. Step (b) of the method can comprise contacting the compound with ⁸⁹Zr-chloride in a hydrochloride solution at a pH in a range of 7 to 9.

Step (c) of the method can comprise purifying the radiolabeled preparation using reverse phase chromatography. Step (c) of the method can comprise purifying the radiolabeled preparation using gradient elution. The gradient elution may use at least two different solvents. One of the solvents can comprise water and trifluoroacetic acid, and another of the solvents can comprise acetonitrile and trifluoroacetic acid.

The labeling agent produced by the method can have a radiochemical stability greater than 60% measured at 72 hours after step (c). In step (d), the method may further comprise adding a stabilizer to the labeling agent. The stabilizer can be ascorbic acid. The labeling agent produced by the method can have a radiochemical stability greater than 80% measured at 72 hours after step (d). In the method, step (b) can comprise creating the radiolabeled preparation at a radiochemical yield of at least 95%, or least 97%.

In another embodiment, the present invention provides a method of labeling of a biological material for imaging. The method comprises contacting a biological material with the labeling agent prepared by the method of the invention such that the biological material becomes labeled for imaging, wherein the biological material is selected from cells, liposomes, DNA aptamers, RNA aptamers, viruses, nanoparticles, microorganisms, antibodies, proteins, peptides, scaffolds, polymers, and nucleic acids. A radiolabeling yield when contacting the biological material with the labeling agent can be at least 5% when the biological material is contacted with the labeling agent at a concentration of the biological material of 0.1 mg/mL. A radiolabeling yield when contacting the biological material with the labeling agent can be at least 15% when the biological material is contacted with the labeling agent at a concentration of the biological material of 0.5 mg/mL. A radiolabeling yield when contacting the biological material with the labeling agent can be at least 30% when the biological material is contacted with the labeling agent at a concentration of the biological material of 1.0 mg/mL.

A first radiolabeling yield when contacting a first amount of the biological material with a first quantity of the labeling agent can be greater than a second radiolabeling yield when contacting a second amount of the biological material with a second quantity of the radiolabeled preparation created in step (b), wherein the first amount and the second amount are the same, and wherein the first quantity and the second quantity are the same. A first radiolabeling yield when contacting a first amount of the biological material with a first quantity of the labeling agent can be at least two times greater than a second radiolabeling yield when contacting a second amount of the biological material with a second quantity of the radiolabeled preparation created in step (b), wherein the first amount and the second amount are the same, and wherein the first quantity and the second quantity are the same.

In yet another embodiment, the present disclosure provides a method for determining radiolabeling efficiency when a biological material is contacted with a labeling agent including a radionuclide to produce a radiolabeled biological material. The method comprises separating the radiolabeled biological material produced when the biological material is contacted with the labeling agent from free radionuclide and unconjugated labeling agent using instant thin layer chromatography. The labeling agent can be the labeling agent prepared by the method of the invention. The biological material can be selected from cells, liposomes, DNA aptamers, RNA aptamers, viruses, nanoparticles, microorganisms, antibodies, proteins, peptides, scaffolds, polymers, and nucleic acids. The instant thin layer chromatography may use an acid-alcohol mixture as a mobile phase and a gel as a solid phase. The acid-alcohol mixture can comprise citric acid and methanol. The gel can comprise silica. In some cases, while the solid phase can be a gel, in other configurations, the solid phase can be a solid (e.g., alumina coated substrate), a liquid supported on a solid, etc.

### EXAMPLES

The following Examples are provided in order to demonstrate and further illustrate certain embodiments and aspects of the present invention and are not to be construed as limiting the scope of the invention.

### Example 1

### Overview of Example 1

Due to the advent of various biologics like antibodies, proteins, cells, viruses, and extracellular vesicles as biomarkers for disease diagnosis, progression, and as therapeutics, there exists a need to have a simple and ready to use radiolabeling synthon to enable noninvasive imaging trafficking studies. Previously, we reported [⁸⁹Zr] zirconium-p-isothiocyanatobenzyl-desferrioxamine ([⁸⁹Zr] Zr-DBN) as a synthon for the radiolabeling of biologics to allow PET imaging of cell trafficking. In Example 1, we focused on improving the molar activity (Aₘ) of [⁸⁹Zr] Zr-DBN, by developing a new reverse phase HPLC method to purify [⁸⁹Zr] Zr-DBN. First, 4.78±0.33 GBq (129.3±8.9 mCi) of ⁸⁹Zr was produced at 40 μA for 180 minutes (3 hours) proton irradiation decay corrected to the end of bombardment with a saturation yield of 4.56±0.31 MBq/μA. Second, after reverse phase HPLC purification, the molar activity of [⁸⁹Zr] Zr-DBN was found to be in 165-316 GBq/μmol range. The high molar activity of [⁸⁹Zr] Zr-DBN also allowed radiolabeling of low concentration of proteins in relatively higher yield. The stability of [⁸⁹Zr] Zr-DBN was measured over time with and without the presence of ascorbic acid. The HPLC method of [⁸⁹Zr] Zr-DBN purification can be adopted in the routine production of [⁸⁹Zr] Zr-DBN as a ready to use labeling synthon.

### Introduction to Example 1

⁸⁹Zr has emerged as a preferred positron emission tomography (PET) isotope for the radiolabeling of various antibodies, viruses, cells, exosomes, extracellular vesicles (EVs) and nanoparticles (NPs) due to its long half-life (78.4 hours) and suitable PET imaging characteristics (β⁺max-0.9 MeV, 22.7%) [Ref. 1-4]. To provide a stable labeling strategy, the macromolecules are normally covalently conjugated with a suitable chelator by using primary amines, hydroxyls or carboxylic groups present on the molecules [Ref. 5]. During the conjugation reaction, appropriately functionalized (activated esters, isothiocyanates, reactive ketones and other easily reactive functional groups) chelators are used in excess (typically 3-6 fold) as compared to the biologics/macromolecules to ensure adequate availability of the chelators on the biologics/macromolecules post-conjugation to enable efficient radiolabeling with ⁸⁹Zr [Ref. 6-13]. The molar activity (Aₘ) of the final radiolabeled biologic/macromolecule depends on the initial starting radioactivity, molar activity of the ⁸⁹Zr, radiolabeling yield and amount (mmol or mg) of the biologics/macromolecules present in the final formulation. To achieve high molar activity (Aₘ), a high concentration of ⁸⁹Zr radioactivity (MBq/μL) is needed, so that only a small mass (μg or ng range) of biologics/macromolecules can be used for an efficient radiolabeling, otherwise more mass of the biologics/macromolecules will be needed to compensate for the dilution caused by the higher volume of radioactivity. The production of ⁸⁹Zr from the cyclotron has been well documented using both liquid targets and solid targets [Ref. 8-10, 13]. Among the various reported methods of ⁸⁹Zr production from solid targets, yttrium foil, yttrium-sputtering, yttrium-pellet and yttrium deposition are commonly known [Ref. 8, 11-12]. In Example 1, we aimed to improve the radiolabeling yield of ⁸⁹Zr labeled biologics/macromolecules by developing a new method of purification of [⁸⁹Zr] zirconium-p-isothiocyanatobenzyl-desferrioxamine ([⁸⁹Zr] Zr-DBN [Ref. 2-3]) as a ready to use radiolabeling synthon for the direct radiolabeling of the biologics/macromolecules with a high molar activity [⁸⁹Zr] Zr-DBN.

### 2. Materials and Methods

### 2.1 Targetry Details

A PETtrace cyclotron (GE HealthCare, Waukesha, WI, USA) was used in this study. To perform Zr-89 production, an Advanced Cyclotron Systems Inc. (ACSI) target holder was used and placed after the switching magnet at a 30 degree angle with respect to beamline on a PETtrace cyclotron; the proton beam energy was degraded using 0.1, 0.2, and 0.3 mm aluminum foils to 15.2, 13.9, and 12.3 MeV, respectively, as estimated from TRIM program. Variable thickness (0.1 mm and 0.127 mm) yttrium foils were purchased form the Alfa-Aesar (50×50 mm, 99.9%) Haverhill, MA, USA.

The trace metal grade concentrated nitric acid (67-70% as HNO₃) and hydrochloric acid (34-37% as HCl) were purchased from the Fisher Chemicals part of the Thermo Fisher Scientific (Waltham MA, USA). Sodium bicarbonate, acetonitrile (HPLC grade) and trifluoroacetic acid (TFA, 99%) were purchased from Sigma-Aldrich (St. Louis, MO, USA). The hydroxamate resin was synthesized as previously described by the Pandey et al. [Ref. 10]. The i-TLC paper was purchased from Agilent Technologies (Palo Alto, CA, USA). The labeling precursor p-SCN-Bn-Deferoxamine (B-705, ≥94%) was purchased from Macrocyclics, Plano, TX, USA. Deionized water was obtained from Barnstead Nanopure water purification system from Thermo Fisher Scientific, Waltham, MA, USA.

### 2.3 Instrumentation

The radioactive samples were counted using a Wizard 2480 gamma counter (Perkin Elmer, Waltham, MA, USA). Radionuclidic purity was evaluated using a high-purity germanium gamma spectrometer (Canberra, Meriden, CT, USA) running Genie 2000 software. The radioactivity readings were recorded using a CRC dose calibrator (489 setting for ⁸⁹Zr and 465 for ⁸⁸Y, CRC-55tPET, Capintec, Ramsey, NJ, USA).

### 2.4 Purification of Cyclotron Produced 89 Zr and Radiosynthesis of [⁸⁹Zr] Zr-DBN

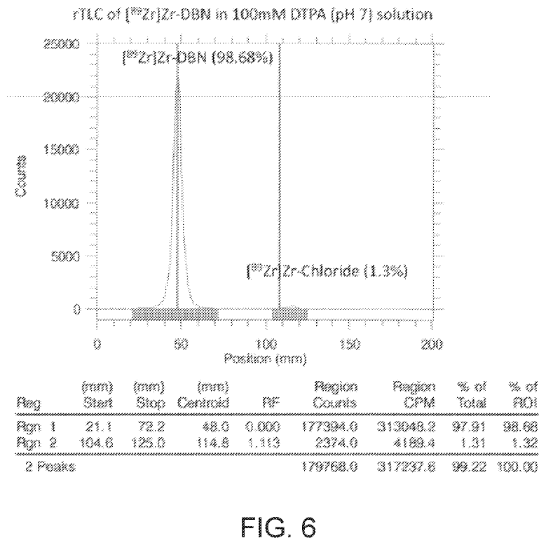

The cyclotron-produced ⁸⁹Zr was purified following previously reported methods [Ref. 8-10]. The purified [⁸⁹Zr] Zr-oxalate was converted to [⁸⁹Zr] Zr-chloride using anion exchange column Chromafix 30-PS-HCO₃ SPE 45 mg cartridge (Macherey-Nagel, Düren, Germany) following the method of Larenkov et al. [Ref. 14]. The cartridge was activated with a 6.0 mL acetonitrile followed by 10 mL saline and 10 mL deionized water wash with air drying steps in between each solvent. The ⁸⁹Zr was trapped on an activated Chromafix 30-PS-HCO₃ SPE (45 mg) cartridge and oxalate was removed with 50 mL deionized water. Finally, ⁸⁹Zr was eluted as [⁸⁹Zr] Zr-chloride with 0.5 mL 1N HCl in a 97.4±1.1 (n=10) elution efficiency. The eluted [⁸⁹Zr] Zr-chloride was then dried using a Savant™ SpeedVac™ High Capacity Concentrator (Thermo Fisher Scientific Inc., Logan, UT, USA) at 0.42 torr and 65° C. The dried [⁸⁹Zr] Zr-chloride was resuspended in ˜180 μL of 0.1N HCl and then neutralized to PH ˜8.0 with ˜18 μL 1M Na₂CO₃. To this, 2.5 mM DFO-Bz-NCS in DMSO (Macrocyclics, Plano, TX, USA) was added to give a final concentration of ˜54 UM DFO-Bz-NCS, and chelation of ⁸⁹Zr proceeded at 37° C. for ˜30 minutes in a thermomixer at 550 rpm. The chelation efficiency was determined by silica gel iTLC (Agilent Technologies Inc., Santa Clara, CA, USA) with 100 mM DTPA PH 7 as the mobile phase. [⁸⁹Zr] Zr-DBN showed an R_{f}=0, whereas [⁸⁹Zr] Zr-chloride had an R_{f}=0.9 (see FIG. 6).

### 2.5 HPLC Purification of [⁸⁹Zr] Zr-DBN

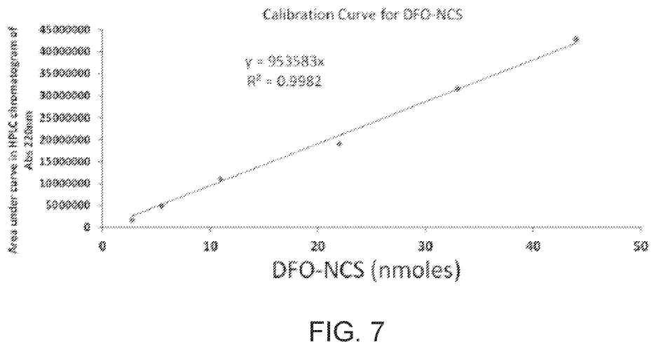

The [⁸⁹Zr] Zr-DBN reaction mixture was diluted to ˜1.0 mL with deionized water (neutralized with sodium carbonate to final pH 7.0) before HPLC purification. The purification was performed at room temperature using a reverse phase Agilent Zorbax 300-SB-C-18 (5 μm; 9.4×250 mm) column (Agilent Technologies Inc., Santa Clara, CA, USA) and 1.0 mL injection loop size. The gradient elution was performed with solvent A (deionized H₂O+0.1% TFA) and solvent B (Acetonitrile+0.1% TFA). The flow rate was set at 1.8 mL/min and absorbance was set at 220 nm. The purification was performed using 0-5 min (static 5% solvent B), 5-10 min (gradient, 5-34% solvent B), 10-95 min (gradient, 34-41.5% solvent B), 95-100 min (gradient, 41.5-85% solvent B), 100-110 min (gradient, 85-5% solvent B) and 110-115 min (static, 5% solvent B) gradient program. The total separation time was ˜35 minutes. Blank runs were performed in between the sample injections. Concentration of nonradioactive (UV) Zr-DBN was estimated using a calibration curve (see FIG. 7).

2.6 Radiolabeling of Antibody with Purified [⁸⁹Zr] Zr-DBN

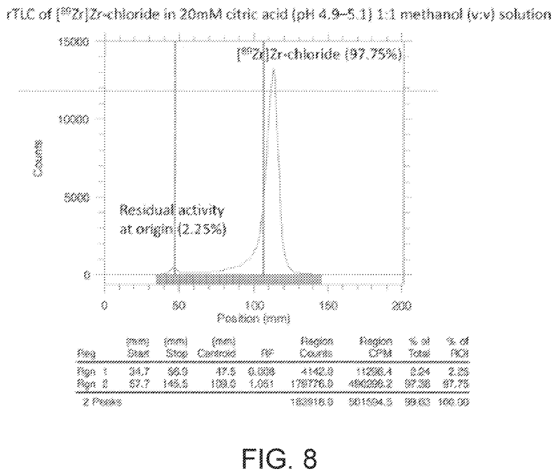

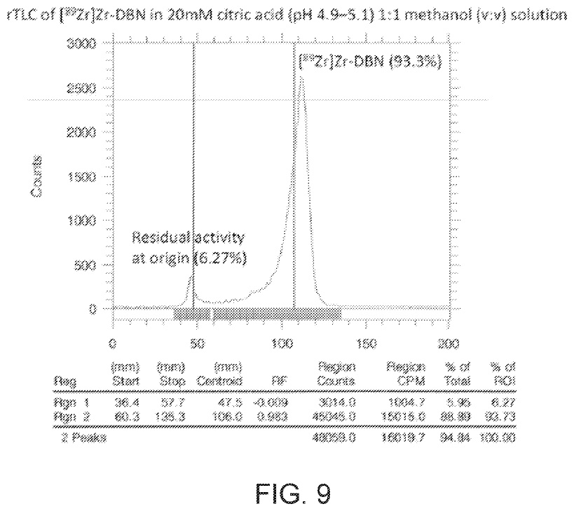

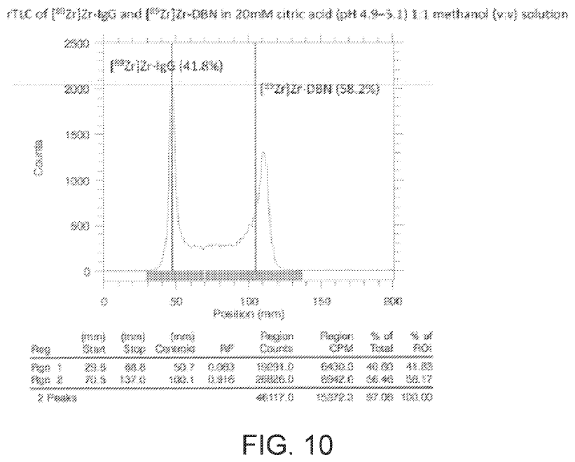

The purified [⁸⁹Zr] Zr-DBN (˜7.2 mL) was collected at the appropriate retention time in a glass test tube and dried using the concentrator at 0.42 torr and at room temperature. For radiolabeling of an example antibody, different concentrations (0.1, 0.5, and 1.0 mg/mL) of human IgG were prepared in phosphate buffered saline (PBS) from a stock solution (˜10 mg/mL) of ChromPure Human IgG, whole molecule (Jackson Immuno Research Inc., West Grove, PA, USA). The pH of the different human IgG solutions was adjusted to pH 9.0 using appropriate volumes of 0.5 M Na₂CO₃. Immediately after adjusting the pH, 200 μL of pH adjusted human IgG solution was added to ˜ 3.7MBq of [⁸⁹Zr] Zr-DBN. The final pH was adjusted with additional volumes of 0.5 M Na₂CO₃ to achieve a pH of 9.0. The IgG radiolabeling reaction was performed at 37° C. for ˜30 minutes in a thermomixer at 550 rpm. After 30 minutes of reaction, the extent of radiolabeling was quantified using silica gel iTLC (Agilent Technologies Inc., Santa Clara, CA, USA) with 20 mM citric acid (pH 4.9-5.1): methanol (1:1, V: V) as the mobile phase. On iTLC, the [⁸⁹Zr] Zr-DBN-IgG showed an R_{f}=0.0, whereas [⁸⁹Zr] Zr-chloride and [⁸⁹Zr] Zr-DBN had an R_{f}=0.99 (see FIGS. 8-10).

### 2.7 Stability of Purified [⁸⁹Zr] Zr-DBN

The purified and concentrated [⁸⁹Zr] Zr-DBN was stored at −20° C. and stability was tested at 0 hours, 24 hours, and 72 hours post HPLC purification and concentration in comparison with unpurified [⁸⁹Zr] Zr-DBN which was also stored at −20° C. To test the stability, the frozen [⁸⁹Zr] Zr-DBN was allowed to come to room temperature and reconstituted with ˜100 μL DMSO. The reconstituted [⁸⁹Zr] Zr-DBN (˜ 3.7 MBq) was diluted with ˜900 μL neutralized deionized water (pH 7.0) and pH was further adjusted to pH 7.0 using 1M Na₂CO₃. The stability of reconstituted and neutralized [⁸⁹Zr] Zr-DBN was tested using the same HPLC method that was used for [⁸⁹Zr] Zr-DBN purification.

### 2.8 Effect of Ascorbic Acid on Stability of Purified [⁸⁹Zr] Zr-DBN

The HPLC purified [⁸⁹Zr] Zr-DBN (˜7.2 mL) was divided into two equal parts of ˜3.6 mL each. In one-part, 25 μL of 200 mg/mL ascorbic acid (Sigma Aldrich, St. Louis, MO, USA) was added. Both ascorbic acid treated, and untreated fractions were concentrated at 0.42 torr and room temperature. The concentrated solutions were stored at −20° C. and stability was tested at 0 hours, 24 hours and 48 hours using the same HPLC method used for [⁸⁹Zr] Zr-DBN purification.

### 2.9. Measurement of Trace Metal Impurity and Radionuclidic Impurities

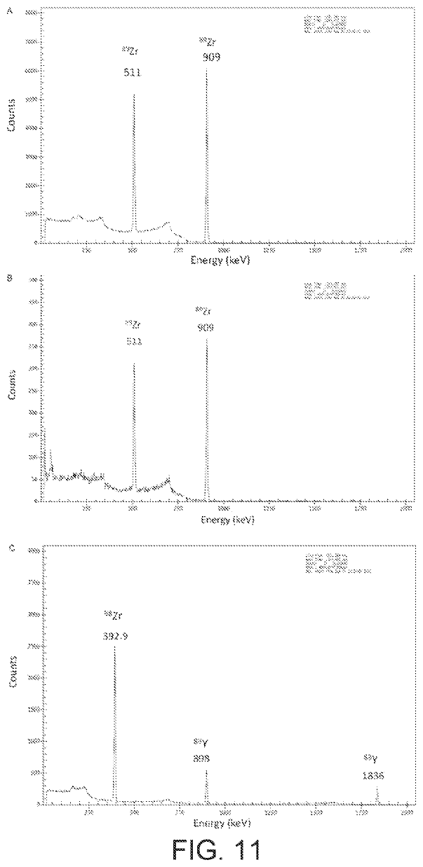

The presence of trace metals (Y, Fe, Al) in the purified samples were analyzed using inductively coupled Plasma spectrometer using PerkinElmer Elan or PerkinElmer NexION 350D ICP-MS spectrometers (Waltham MA, USA) equipped with Elemental Scientific Inc. (ESI) SC2-DX autosamplers (Omaha NE, USA). The mass spectrometers were equipped with ESI microflow PFA-ST nebulizers and quartz cyclonic spray chambers including a baffle. Radionuclidic purity was measured via high purity germanium gamma spectrometer (HPGe). Purified [⁸⁹Zr] Zr-oxalate samples were counted in a dose calibrator (at Zr-89 setting, 489) and analyzed by HPGe, at different time points over the period of 3-4 months from the day of purification to estimate the presence of ⁸⁸Zr (T_{1/2} 83.4 days) due to possible 89Y (p, 2n) ⁸⁸Zr reaction and its daughter radionuclide ⁸⁸Y (T_{1/2} 106.6 days) in the final formulation (FIG. 11, panels A-C). Using the manufacturer's ionization response versus gamma energy of the dose calibrator (Capintec/Mirion, Florham Park, NJ, USA), the radioactivities of ⁸⁸Zr and ⁸⁸Y were estimated at time of measurement, and decay corrected to estimate 88Zr produced at EOB.

### 2.10. Statistical Analysis

All values are given as mean±standard deviation (SD). Statistical significance of differences was determined by two-tailed student's T-test. P values<0.05 were considered statistically significant.

### Results and Discussion

3.1. Production of ⁸⁹Zr from Cyclotron Using Solid Target

To enhance our cyclotron production capability of ⁸⁹Zr, we switched our production method from liquid target to solid target [Ref. 9-10]. For the simplicity of ⁸⁹Zr production, we chose to use a yttrium foil as a target material. To optimize ⁸⁹Zr production yield, we tested various thicknesses of yttrium foil (0.1-0.25 mm), different beam current (25-40 μA) and different irradiation durations (120-180 minutes). The production yields are listed in Table 1. We noticed an expected 2.33-fold improvement in saturation yield from 2.07±0.60 MBq/μA to 4.84±0.88 MBq/μA on changing yttrium foil thickness from 0.1 mm to 0.25 mm along with beam current from 25-30 μA to 40 μA for a 120-minute proton irradiation. To reduce overall yttrium content in our final solution, the ⁸⁹Zr production yield was optimized with 0.2 mm thick yttrium foil (two foils of 0.1 mm thickness) at 40 μA beam current for 180 minutes of irradiation. We found a saturation yield of 4.56±0.31 MBq/μA with radioactivity of 4.78±0.33 GBq (129.3±8.9 mCi) of ⁸⁹Zr, decay corrected to end of the beam. Following previously developed methods, irradiated yttrium foil was dissolved slowly in 2 mL of 6N-HNO₃ at room temperature. After complete dissolution, the solution was diluted with 7 mL of deionized water and loaded slowly onto the hydroxamate resin (100 mg) column. After loading, the hydroxamate resin was washed with 20 ml of 2N-HCl to remove trace quantities of yttrium salt followed by 10 mL of deionized water to remove any leftover acid before eluting ⁸⁹Zr with 3.0 mL of 1M oxalic acid.

TABLE 1
Cyclotron production of ⁸⁹Zr

| | | Yttrium | | | |
|---|---|---|---|---|---|
| Beam | Irradiation | foil | Corrected | Corrected | |
| current | time | thickness | yield at EOB* | yield at EOB | Saturation yield |
| (μA) | (min) | (mm) | (mCi) | (MBq) | (GBq/μA) |
| 25-30 (n = 6) | 120 | 0.1 | 27.1 ± 7.8 | 1001 ± 288 | 2.07 ± 0.60 |
| 40 (n = 8) | 120 | 0.25 | 91.8 ± 16.8 | 3396 ± 620 | 4.84 ± 0.88 |
| 40 (n = 19) | 180 | 0.2 | 129 ± 8.9 | 4784 ± 330 | 4.56 ± 0.31 |

*EOB: End of bombardment

The purified ⁸⁹Zr solution was analyzed for the presence of trace quantities of Y, Zr, Fe and Al using MP-AES and results showed no unexpected levels of any of tested metal ions. In addition to metal ion impurity, we also tested the radionuclidic purity using HPGe for the presence of ⁸⁸Zr or its daughter nuclei ⁸⁸Y. Based on our analysis, we found 0.014±0.006% (n=5) of total non ⁸⁹Zr related radioactivity as a radionuclidic impurity at end of purification, which includes both ⁸⁸Zr due to potential (p,2n) reaction and ⁸⁸Y (T_{1/2} 106.6 days) the daughter nuclei of ⁸⁸Zr (T_{1/2} 83.4 days).

### 3.2 Radiosynthesis and HPLC Purification of [⁸⁹Zr] Zr-DBN

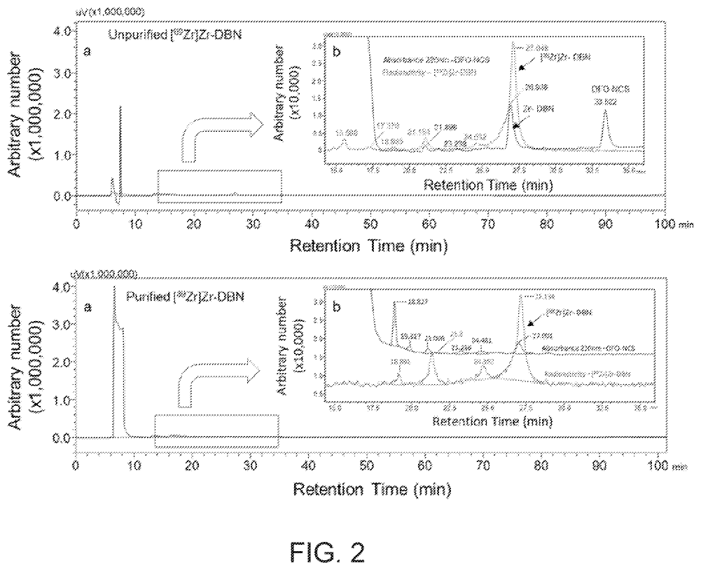

The [⁸⁹Zr] Zr-DBN was synthesized as reported previously by reacting [⁸⁹Zr] Zr-chloride (neutralized to PH ˜8.0) and 10.7 moles of DFO-Bz-NCS in DMSO at 37° C. for ˜30 minutes. The percentage of radiolabeling was determined by silica gel iTLC using 100 mM DTPA (pH 7) as the mobile phase. Based on iTLC, radiolabeling yield was found to be >95%. In previous studies, we used unpurified [⁸⁹Zr] Zr-DBN for labeling of various biologics (proteins, cells, viruses, EVs, etc.) but noticed some biologics (having low protein concentration) were more sensitive and gave poor radiolabeling yield with unpurified [⁸⁹Zr] Zr-DBN. Therefore, in Example 1, we attempted to purify [⁸⁹Zr] Zr-DBN to remove unlabeled p-SCN-Bn-desferoxamine (DBN) and also to increase molar activity (Aₘ) of [⁸⁹Zr] Zr-DBN to enhance radiolabeling yield of biologics having low protein concentration with the purified [⁸⁹Zr] Zr-DBN. To purify [⁸⁹Zr] Zr-DBN, initially, we attempted various solid phase cartridges to separate [⁸⁹Zr] Zr-DBN with DBN but in vain. We evaluated reverse phase HPLC (Zorbax 300-SB-C-18.5 μm; 9.4×250 mm) to separate [⁸⁹Zr] Zr-DBN with DBN, and after trying various solvents and their combinations, we finally settled on a gradient elution method, which was comprised of solvent A (deionized water with ±0.1% TFA) and solvent B (Acetonitrile+0.1% TFA) with a flow rate of 1.8 mL/min as described in methods section above. Using this newly developed HPLC method, we successfully separated [⁸⁹Zr] Zr-DBN with DBN (see FIG. 2). The unlabeled DBN (DFO-NCS) eluted at the retention time of 33.5 minutes, whereas labeled [⁸⁹Zr] Zr-DBN eluted at 27.1 minutes, showing good separation. The [⁸⁹Zr] Zr-DBN was collected and concentrated (SpeedVac) to remove acetonitrile and trifluoroacetic acid before using it for the radiolabeling of the antibody/protein (IgG). The molar activities of purified and unpurified [⁸⁹Zr] Zr-DBN were measured and presented in Table 2. It is important to mention here that the same concentration of (mg/mL) of labeling protein (IgG), starting radioactivity (mCi or MBq) of [⁸⁹Zr] Zr-DBN and same labeling conditions (pH, temperature and reaction time) were used to radiolabel protein (IgG) with both purified and unpurified [⁸⁹Zr] Zr-DBN. To avoid any potential confound from the starting ⁸⁹Zr, we always used same batch of cyclotron produced and purified ⁸⁹Zr for the synthesis of [⁸⁹Zr] Zr-DBN and antibody labeling experiments with or without the [⁸⁹Zr] Zr-DBN HPLC purification step.

3.3 Radiolabeling of Antibody (IgG) with HPLC Purified [⁸⁹Zr]Zr-DBN

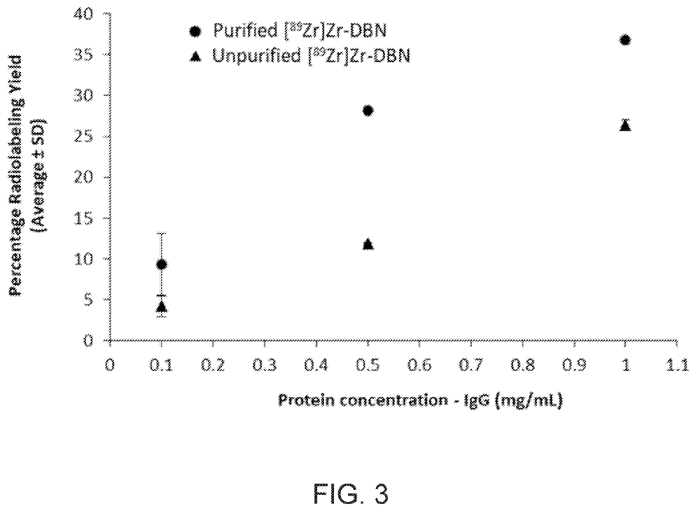

The radiolabeling of IgG was performed at various concentrations of antibody (0.1-1.0 mg/mL) to study radiolabeling efficiency as a function of conjugatable protein concentration. To measure the radiolabeling efficiency, we developed a new iTLC system in which both free ⁸⁹Zr and unconjugated [⁸⁹Zr] Zr-DBN were separated from the radiolabeled protein [⁸⁹Zr] Zr-DBN-IgG. The system employs 20 mM citric acid (pH 4.9-5.1), methanol (1:1, v/v) as a mobile phase and silica gel iTLC as the solid phase. In this system, we independently confirmed the R is of [⁸⁹Zr] Zr-chloride and [⁸⁹Zr] Zr-DBN to be 0.99 (solvent front) and 0.0 (origin) for radiolabeled IgG protein ([⁸⁹Zr] Zr-DBN-IgG, see FIGS. 8-10). The purified [⁸⁹Zr] Zr-DBN gave 2.5 fold higher radiolabeling yield than unpurified [⁸⁹Zr] Zr-DBN at 0.1 mg/mL concentration of protein (IgG), and a similar trend of 2.4 fold higher radiolabeling yield was noted for 0.5 mg/mL concentration of IgG protein (IgG) (Table 2, FIG. 3). However, we noticed a lower impact of HPLC purification of [⁸⁹Zr] Zr-DBN on radiolabeling yield for 1.0 mg/mL concentration of IgG protein (1.4 fold higher yield with purified [⁸⁹Zr] Zr-DBN) (Table 2). These data suggest that the beneficial effect of purification of [⁸⁹Zr] Zr-DBN on labeling yield is dependent on protein concentration with more benefit for labeling of biologics having low protein concentrations (see FIG. 3).

TABLE 2
Comparison of Radiolabeling Yield of Protein (IgG) with Molar
Activity (Aₘ) of Purified and Unpurified [⁸⁹Zr]Zr-DBN

| | | % | | | | Average fold |
|---|---|---|---|---|---|---|
| | | Radiolabeling | | % | | change in |
| | Molar | yield of | Molar | Radiolabeling | Average fold | radiolabeling |
| | activity of | protein (IgG) | activity of | yield of | change in | yield of |
| | unpurified | With | purified | protein (IgG) | molar | protein (IgG) |
| | [⁸⁹Zr]Zr-DBN | unpurified | [⁸⁹Zr]Zr-DBN | with purified | activity of | with purified |
| Protein | (GBq/μmol) | [⁸⁹Zr]Zr-DBN | (GBq/μmol) | [⁸⁹Zr]Zr-DBN | purified over | over |
| concentration | Average ± | Average ± | Average ± | Average ± | unpurified | unpurified |
| (mg/mL) | SD | SD | SD | SD | [⁸⁹Zr]Zr-DBN | [⁸⁹Zr]Zr-DBN |
| 0.1 (n = 4) | 20.62 ± 3.99 | 3.85 ± 1.23 | 165.67 ± 133.44 | 10.54 ± 3.06* | 8.0 | 2.7 |
| 0.5 (n = 2) | 24.98 | 11.91 ± 0.06 | 316.31 | 28.18 ± 0.31* | 12.7 | 2.4 |
| 1.0 (n = 3) | 22.36 ± 37.00 | 26.43 ± 0.61 | 189.30 ± 179.61 | 36.76 ± 0.03* | 8.4 | 1.4 |

*P value < 0.05 with respect to unpurified [⁸⁹Zr]Zr-DBN

### 3.4 Stability of Purified [⁸⁹Zr] Zr-DBN Overtime and Effect of Ascorbic Acid

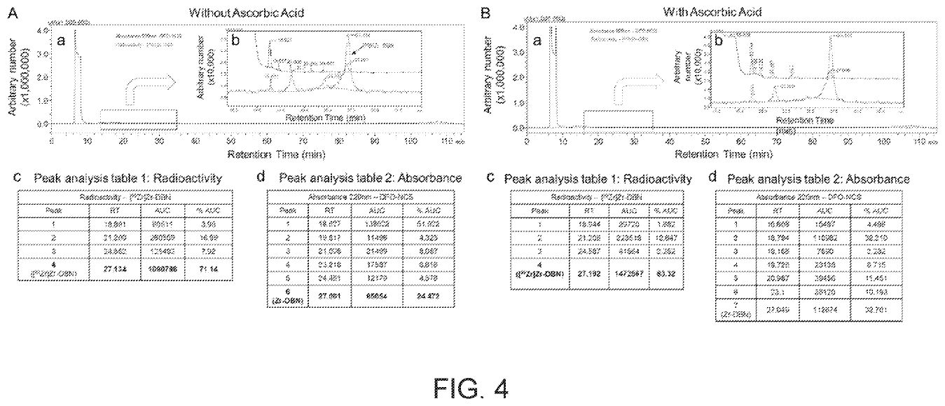

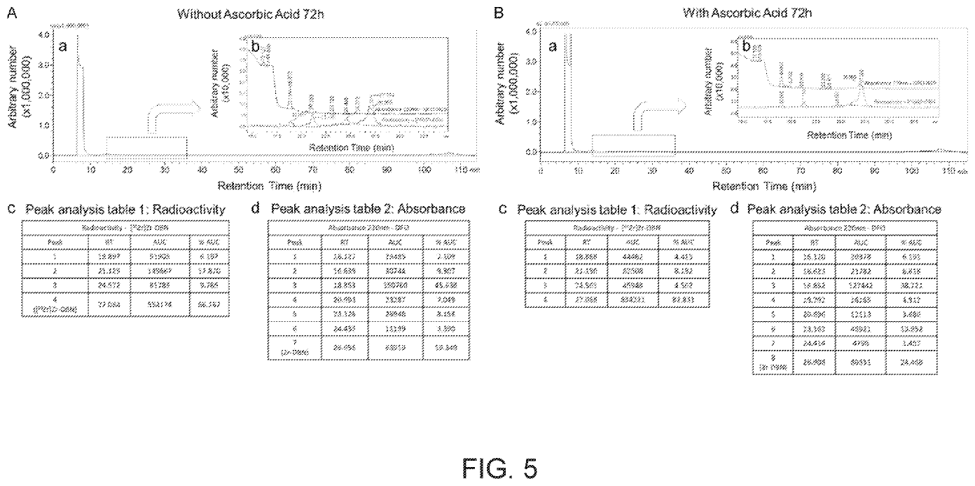

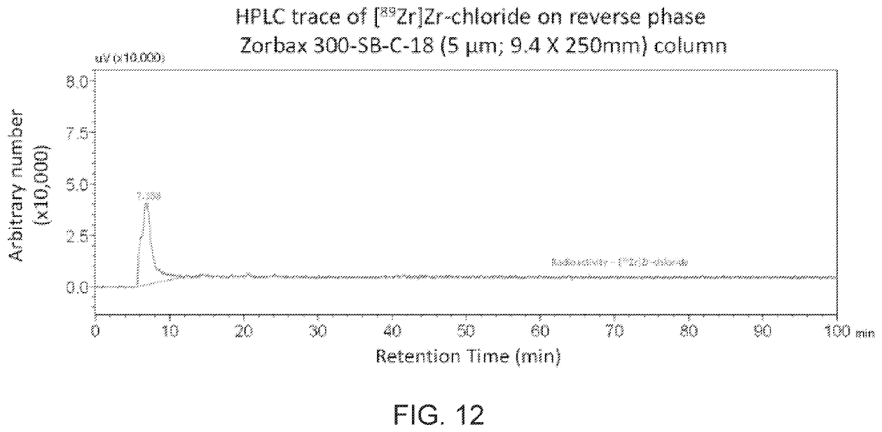

Encouraged by high protein (IgG) radiolabeling yield with the purified [⁸⁹Zr] Zr-DBN, especially at lower protein concentrations, we thought to evaluate the radiochemical stability of [⁸⁹Zr] Zr-DBN over time following HPLC purification and concentration. We noticed appearance of additional small radioactivity peaks at 18.8 minutes, 21.2 minutes and 24.5 minutes retention time other than the expected 27.1 minutes peak for the [⁸⁹Zr] Zr-DBN on HPLC analysis immediately after concentrating the collected HPLC fractions for the [⁸⁹Zr] Zr-DBN (FIG. 4, panel a). Based on the retention time of the additional peaks, it was evident that none of the additional peaks resulted from demetallation of ⁸⁹Zr since free ⁸⁹Zr appears at ˜6 minutes retention time on the HPLC analysis (see FIG. 12). Therefore, based on our experience, we suspected, it could be due to the radiation induced decomposition of [⁸⁹Zr] Zr-DBN [Ref. 15]. Therefore, without further characterizing newly appeared peaks on HPLC, we treated the collected HPLC fraction with 25 μL of 200 mg/mL ascorbic acid before the concentration step [Ref. 15]. As expected, the addition of this small quantity of ascorbic acid did help and reduced the total areas under the curve for the additional peaks present at 18.8 minutes, 21.2 minutes and 24.5 minutes retention times while enhancing the 27.1 minutes peak for [⁸⁹Zr] Zr-DBN by ˜12% (FIG. 4, panel b). Addition of ascorbic acid improved stability of [⁸⁹Zr] Zr-DBN at 72 hours from 66% to >82% (FIG. 5, panels a & b). We noted that the relative percentage of radioactivity peaks in some of the fractions were not changed even after the concentration step while others showed less protection with ascorbic acid.

### Conclusions

In Example 1, we produced high quantities of ⁸⁹Zr (4783±330 MBq, 129.3±8.9 mCi) with a saturation yield of 4.56±0.31 MBq/μA using yttrium foil via proton irradiation, and then successfully developed a reverse phase HPLC method for the purification of [⁸⁹Zr] Zr-DBN and a new iTLC system for instant monitoring of radiolabeling yield of antibodies with [⁸⁹Zr] Zr-DBN. The successful production of high molar activity of [⁸⁹Zr] Zr-DBN has allowed 1.4-2.7 fold higher radiolabeling yield at various concentrations of antibody protein (0.1-1.0 mg/mL) in comparison to unpurified (low molar activity) [⁸⁹Zr] Zr-DBN. The long-term (up to 72 hours) stability of purified [⁸⁹Zr] Zr-DBN was also studied with and without addition of ascorbic acid. Thus, present Example 1 enables the translation of [⁸⁹Zr] Zr-DBN as a ready to use synthon for on-demand and on site radiolabeling of various biologics.

### Example 2

### Overview of Example 2

In Example 2, we investigated the direct cell labeling and tracking of white blood cells and stem cells in healthy athymic mice by evaluating different ⁸⁹Zr-labeled synthons. Cell based therapies are evolving as an effective new approach to treat various diseases. To understand the safety, efficacy and mechanism of action of cell-based therapies, it is imperative to follow their biodistribution noninvasively. The positron-emission-tomography (PET) based non-invasive imaging of cell trafficking offers such a potential. In Example 2, we evaluated a ready to use direct cell radiolabeling synthon-[⁸⁹Zr] Zr-DFO-Bn-NCS for noninvasive PET imaging based trafficking of white blood cells (WBCs) and stem cells (SCs) up to 7 days in athymic nude mice. We compared the degree of ⁸⁹Zr complexation and percentage of cell radiolabeling efficiencies for the synthon. The synthon [⁸⁹Zr] Zr-DFO-Bn-NCS, was successfully prepared, and used for radiolabeling of WBCs and SCs. A relatively high cell radiolabeling yield was found for [⁸⁹Zr] Zr-DFO-Bn-NCS. In terms of biodistribution, WBCs radiolabeled with [⁸⁹Zr] Zr-DFO-Bn-NCS primarily accumulated in liver and spleen. This study offers an appropriate selection of ready to use radiolabeling synthon for a noninvasive trafficking of WBCs, SCs and other cell-based therapies.

### Introduction to Example 2

Safety and efficacy are the two main pillars of any therapeutics, cell-based therapies and imaging are no exception, however not much is known to effectively assess the biodistribution, clearance and efficacy of cell-based therapies due to the absence of an appropriate noninvasive imaging tool. In vivo cell tracking could provide information about distribution, localization, and clearance of various cell-based therapies including immune cell (CAR-T cells), stem cells and hepatocytes post-administration in the body. There are various non-invasive molecular imaging modalities that could be employed to track cell based therapies including optical imaging via fluorescence imaging (FLI) [Ref. 16-17], bioluminescence imaging (BLI) [Ref. 18-19], and ultrasound-guided photoacoustic imaging (PA) [Ref. 20-22]. The magnetic resonance imaging (MRI) [Ref. 23-25] and radiology imaging such as computed tomography (CT) [Ref. 26-28] and nuclear medicine imaging such as positron emission tomography (PET) [Ref. 29-33], and single photon emission computed tomography (SPECT) [Ref. 34-35] could also be employed to effectively measure the distribution, localization, and clearance of various cell-based therapies overtime and to shed light on safety and efficacy of the cell based therapies.

Among various imaging modalities, optical imaging modality is restricted to small animals due to limited tissue penetration (1-2 mm) in humans. Whereas MRI and CT provide high resolution anatomical information; but have low sensitivity in both animals and humans. Both PET and SPECT are advantageous over other techniques and often integrated with CT and MRI. The PET/CT or SPECT/CT or PET/MRI provides quantitative and temporal distribution of immune and stem cells in animals and patients with no limitation of tissue penetration due to high energy gammas [Ref. 36-39].

Cells can be radiolabeled either directly or indirectly [Ref. 40]. The direct cell radiolabeling consists of ex-vivo radiolabeling of cells prior to their administration into body followed by short-term (<7 days) in vivo tracking of these radiolabeled cells. The potential limitation of direct cell labeling approach is the short-term tracking capability due the decay of the radioactivity over time and or efflux of radiotracer or instability of labeled radioactive tag over time. On the other hand, indirect cell radiolabeling method is based on transfection of reporter gene (e.g., sodium iodide symporter (NIS) [Ref. 41], green fluorescent protein (GFP), simplex herpes virus type-1 thymidine kinase (HSV1-tk) etc.) in the cells that selectively takes up the respective radioactive or non-radioactive reporter probe in the cells upon exposure to its respective reporter probe. If the administered cells keeps expressing reporter protein after administration, then repeated systemic administration of its reporter probe allows long term-visualization of administered cells. Although indirect cell labeling approach allows long term visualization of administered, genetic modification for cell labeling remains a regulatory hurdle.

Cell radiolabeling using the direct cell radiolabeling approach with various SPECT radiopharmaceuticals such as [^{99m}Tc] Tc-HMPAO (t_{1/2}=6.01 hours) [Ref. 42-44], and [111|n] In-oxine (t_{1/2}=68.2 hours) [Ref. 45-48] have been used to track leukocytes for infection and inflammation imaging over past four decades. SPECT is a powerful clinical imaging tool with lower usage cost than PET due to the lack of need of an onsite cyclotron. However, 2-3-fold higher sensitivity of PET over SPECT, superior spatial resolution of PET in clinical setting and quantitative nature makes it a preferred imaging modality for tracking a single cell or small number of administered radiolabeled cells with more precise quantification and hence, require lower radiation exposure [Ref. 49]. Examples of commercial PET probes used to label the cells includes [18F]-FDG (t_{1/2}=109.7 min, 3±=97%) [Ref. 50], [64Cu] Cu-PTSM (t_{1/2}=12.7 hours, β⁺=17.9%) [Ref. 51] and [68Ga] Ga-oxine (t_{1/2}=68 min, β⁺=88.8%) [Ref. 52-53].

Recently, among PET radioisotopes, zirconium-89 (3±=22.3%) is gaining popularity for tracking cells attributed to well established cyclotron-mediated production, longer half-life of 3.27 days and low average positron energy (E_{β}+=0.389 MeV) which enables monitoring of radiolabeled cells either through direct cell labeling (also called non-specific cell labeling agents) [Ref. 54-55] or indirect labeling approach mediated through antibodies [Ref. 56-57], peptides [Ref. 58], proteins [Ref. 59] and nanoparticles [Ref. 60-62] at late time points (up to 3-weeks).

Among various chelators used for the radiolabeling of cells with ⁸⁹Zr are tropolone, malonate, hydroxamates, and oxine (8-hydroxyquinoline). Among these, oxine forms a lipophilic complex with ⁸⁹Zr and enters in the cells passively. To date, [⁸⁹Zr] Zr-oxine is commonly used radiotracer to label various cells including tumor cell lines [Ref. 63-64], bone marrow [Ref. 65-66] T cells [Ref. 67], NK cells [Ref. 68], WBCs [Ref. 69], stem cells [Ref. 70] and leukocytes [Ref. 71]. However, efflux of ⁸⁹Zr from cells labeled with [⁸⁹Zr] Zr-oxine remains a challenge. Recently, Friberger et al. reported one-step clinically translatable method of synthesis of [⁸⁹Zr] Zr-oxine and cell labeling efficiency of (61-68%) with human decidual stromal cells (hDSCs), bone marrow-derived macrophages (rMac) and human peripheral blood mononuclear cells (hPBMCs), however, 29-38% apparent efflux of ⁸⁹Zr from the labeled cells raised a further concern of radiotoxicity and non-specificity of signal [Ref. 72].

Besides [⁸⁹Zr] Zr-oxine, the other reported method of cell labeling was covalent attachment of radiolabeled [⁸⁹Zr] Zr-DFO-Bn-NCS complex with the primary amines present on the cell surface proteins to form stable thiourea bond, which has solved the efflux problem observed with [⁸⁹Zr] Zr-oxine [Ref. 2, 3, 72, and 73]. The [⁸⁹Zr] Zr-DFO-Bn-NCS has been successfully used to radiolabel mouse melanoma, mouse dendritic cells and human mesenchymal stem cells with insignificant efflux of free ⁸⁹Zr from [⁸⁹Zr] Zr-DFO-Bn-NCS overtime (7 days-post radiolabeling) [Ref. 2]. Additionally, a better version of DFO chelator as DFO* has been developed to further strengthen the stability of ⁸⁹Zr complexation and showed lower bone uptake over time [Ref. 74]. Various other chelators are also being developed to address in vivo stability of ⁸⁹Zr complex over time [Ref. 75-79].

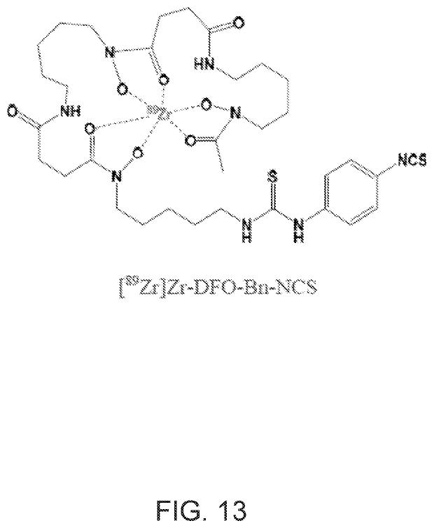

In Example 2, we have optimized and compared the radiolabeling yields of white blood cells (WBCs) and stem cells (SCs) using a ready to use labeling synthon [⁸⁹Zr] Zr-DFO-Bn-NCS [Ref. 2, 3, 72, and 73] (see FIG. 13), and evaluated its applications in cell trafficking to better understand the biodistribution/pharmacokinetics of cell based therapies. This approach can be extended to various other cell-based therapies like CAR-T cell therapy.

### Materials and Methods

General. The ⁸⁹Zr used in this study was produced on a PETtrace cyclotron (GE Healthcare, Waukesha, WI, USA) using 89Y target foil (0.1 mm; 50×50 mm, 99.9%), which was purchased from Alfa-Aesar, Haverhill, MA, USA. The trace metal grade nitric acid (67-70%), hydrochloric acid (34-37%) were purchased from Thermo Fisher Scientific, Waltham, MA, USA. Sodium bicarbonate, oxalic acid dehydrate (TraceSELECT®>99.9999% metal basis), sodium carbonate, sodium citrate dihydrate and HPLC grade acetonitrile were purchased from Sigma Aldrich, St. Louis, MO, USA. The silica gel iTLC were purchased from Agilent Technologies, Santa Clara, CA, USA. The chelator p-SCN-Bn-Deferoxamine or DFO-Bn-NCS (≥94%) was purchased from Macrocyclics, Plano, TX, USA. The empty Luer-Inlet SPE cartridges (1 mL) with frits (20 μm pore size) were purchased from (Supelco Inc, Bellefonte, PA, USA) and Chromafix® 30-PS-HCO₃ PP cartridge (45 mg) were purchased from Macherey-Nagel, Duren, Germany. The Millex®-GV filter (0.2 μm) was purchased from Millipore Sigma, Burlington, MA, USA. The hydroxamate resin used in this study was synthesized as demonstrated by Pandey et al. [Ref. 9, 10, 13, 80]. Thermomixer was purchased for Eppendorf, Hamburg, Germany.

Production and Purification of [⁸⁹Zr] ZrCl₄

The ⁸⁹Zr was produced using yttrium foil on a solid target through a ⁸⁹Y (p,n) ⁸⁹Zr nuclear reaction in PETtrace cyclotron as described previously by Pandey et al. [Ref. 80 and Example 1 above]. ⁸⁹Zr was purified first as [⁸⁹Zr] Zr-oxalate and then converted to [⁸⁹Zr] ZrCl₄ using activated Chromafix 30-PS-HCO₃ SPE as demonstrated by Pandey [Ref. 9, 10, 13, 80 and Example 1 above] and Larenkov et al. [Ref. 14], respectively. The final [⁸⁹Zr] ZrCl₄ was eluted in ˜0.5 mL of 1.0 N HCl and then dried using a steady flow of nitrogen gas in a V-vial at 65° C.

Apparent Molar Activity of [⁸⁹Zr] ZrCl₄

The apparent molar activity of ⁸⁹Zr was estimated using a DFO-Bn-NCS titration method. In this method, 10 μL [⁸⁹Zr] Zr-chloride (36.4 MBq) was added to 90 μL de-ionized H₂O. To this, 4 μL of 0.5 M Na₂CO₃ was added to neutralize and adjust the pH to 7.5-8.0. To the neutralized mixture, 0.01-10 μg of DFO-Bn-NCS in 4 μL of DMSO was added and mixed. The complexation mixture was then incubated at 37° C. for 1 hour. After 1 hour, the degree of ⁸⁹Zr complexation was determined with respect to the DFO-Bn-NCS concentration using radio-TLC with 20 mM sodium citrate (pH 4.9-5.1) as a mobile phase. The complexed ⁸⁹Zr as [⁸⁹Zr] Zr-DFO-Bn-NCS showed at the origin with R_{f}=0, whereas free or un-complexed ⁸⁹Zr had an R_{f} of 0.99 (solvent front). The half maximal inhibitory concentration (IC₅₀) of DFO-Bn-NCS in mg/mL was calculated using non-linear regression curve fitting analysis with two equations-Equation 1: [Concentration] vs Complexation (three parameter) and Equation 2: log (concentration) vs. complexation (three parameter). The analysis was performed using analysis software-GraphPad Prism 9 (GraphPad Software, San Diego, CA, USA). The minimum ligand concentration for which 100% complexation occurred was estimated by multiplying the IC₅₀ by 2, and the apparent molar activity (GBq/μmole) and the apparent specific activity (GBq/mg) of ⁸⁹Zr were calculated by correcting for the total activity divided by μmoles or mg of DFO-Bn-NCS needed for 100% ⁸⁹Zr complexation.

### Radiosynthesis of [⁸⁹Zr] Zr-DFO-Bn-NCS,

The radiosynthesis of the different synthons [⁸⁹Zr] Zr-DFO-Bn-NCS, was performed using a modified procedure demonstrated in our previous work [Ref. 2]. The purified [⁸⁹Zr] Zr-chloride was resuspended in appropriate volume of 0.1 N HCl and then neutralized to PH ˜8.0 with 0.5 M Na₂CO₃. The neutralized [⁸⁹Zr] Zr-chloride solution (70-100 μL) containing ˜21 MBq of ⁸⁹Zr was used in the case of DFO-Bn-NCS. To this neutralized [⁸⁹Zr] Zr-chloride solution, 4 nmoles of DFO-Bn-NCS (prepared in DMSO) were added in separate reactions. The resultant reaction mixtures were stirred for 30 minutes at 37° C. in a thermomixer at 500 rpm. The radiolabeling efficiency was determined at different time points by silica radio-TLC using 20 mM sodium citrate (pH 4.9-5.1) as a mobile phase.

### Cell Preparation

The human mesenchymal stem cells (SCs) were from the Department of Cardiovascular Medicine, Mayo Clinic, Rochester, MN, USA, and white blood cells (WBCs) were isolated from the peripheral blood provided by the Division of Transfusion Medicine, Mayo Clinic, Rochester, MN, USA. The isolation of WBCs from the peripheral blood was performed using Lymphoprep™ (STEMCELL Technologies Inc., Canada) gradient centrifugation method as per manufacturer instruction. The final WBS solution was washed with Hank's Balanced Salt Solution.

### Cell Radiolabeling

The SCs and WBCs cells were radiolabeled with, [⁸⁹Zr] Zr-DFO-Bn-NCS. The cell radiolabeling mixture was prepared by mixing equal volume of the [⁸⁹Zr] Zr-DFO-Bn-NCS reaction mix and equal volume of phosphate buffer-HEPES. The phosphate buffer-HEPES was prepared by mixing 120 μL of 1.2 M phosphate buffer and 100 μL of 1M HEPES. This cell radiolabeling mix was incubated at room temperature for 30 minutes. After this incubation, the cell radiolabeling mixture (˜150-200 μL) was added to cell suspension which was at a concentration of 6×10⁶ cells in 500 μl HEPES Buffered Hank's Balanced Salt Solution at pH 7.5-8.0 [Ref. 2-74]. The cell radiolabeling was performed for 30 minutes at room temperature for WBCs and 37° C. for SCs in a thermomixer. Post-radiolabeling the cells were washed 3× with complete Dulbecco's Modified Eagle Medium.

### Trypan Blue Exclusion Assay Cellular Viability Test

The effect of radiolabeling on cellular viability was assessed using trypan blue exclusion assay test within 1 hour of labeling.

### Animals

8-10 weeks old Athymic nude mice (male and female, 1:1) were obtained from Charles Rivers Laboratories or Taconic Biosciences, Inc.

### PET Imaging and Ex Vivo Biodistribution Studies

After radiolabeling, the radiolabeled WBCs-0.1-0.6×10⁶ (0.03-0.11 MBq); and SCs-0.1-1×10⁶ (0.1-0.15 MBq) were injected in a group (n=3) of athymic nude mice, via tail vein injection. PET images were acquired at 4 hours, 2 days, 4 days and 7 days post-injection (p.i.) using microPET scanner. The free [⁸⁹Zr] ZrCl₄ with radioactivity (0.15-0.19 MBq) was also injected intravenously via tail vein injection. The micro PET images were analyzed and scaled to SUV by image analysis software. The animals were euthanized at 7 days p.i., and organs/tissues were collected to measure standardized uptake value (SUV) in major organs. Animals were euthanized via cardiectomy under anesthesia using isoflurane as approved by the Institutional Animal Care and Use Committee (IACUC) of the Mayo Clinic Rochester MN, USA.

### Statistics

The obtained data were analyzed using Microsoft Excel® program and results were compared using unpaired Student's t-test analysis. Differences were regarded as statistically significant for p<0.05.

### Results and Discussion

Production of [⁸⁹Zr] ZrCl₄ and Radiosynthesis of [⁸⁹Zr] Zr-DFO-Bn-NCS

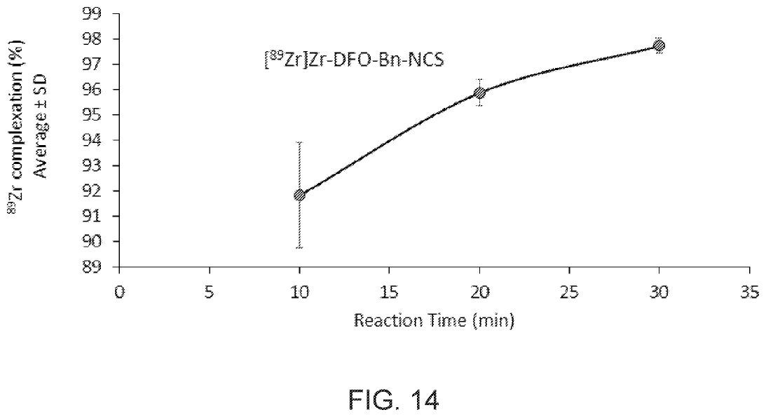

The PET isotope ⁸⁹Zr was produced and purified using cyclotron as described earlier by Pandey et al. [Ref. 80 and Example 1 above] in a high apparent molar activity of 17.0-23.13 GBq/μmol, as assessed by complexing purified [⁸⁹Zr] ZrCl₄ with different amounts of DFO-Bn-NCS. The synthon DFO-Bn-NCS was successfully conjugated with ⁸⁹Zr at 37° C.; pH 7.5-8.0 for 30 minutes in 72-98% radiolabeling yield. The DFO-Bn-NCS showed complexation yield of 97.76±0.31% (n=3) (see Table 3, FIG. 14).

TABLE 3
Percentage of ⁸⁹Zr complexation with DFO-Bn-NCS,
at different reaction times.

| | | Percentage of ⁸⁹Zr complexation |
|---|---|---|
| | | with different synthons |
| Labeling | Reaction | (Average ± SD) |
| Condition | Time | [⁸⁹Zr]Zr-DFO-Bn-NCS (n = 3) |
| 37° C., | 10 min | 91.83 ± 2.07 |
| pH 7.5-8.0 | 20 min | 95.89 ± 0.51 |
| | 30 min | 97.76 ± 0.31 |

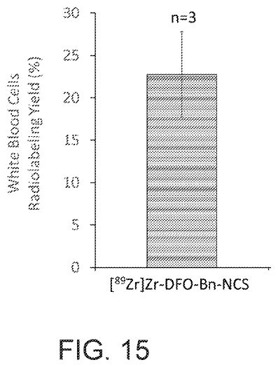

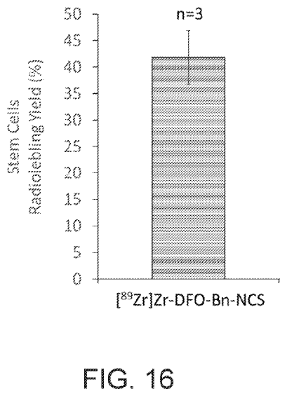

### Radiolabeling of WBCs and SCs

[⁸⁹Zr] Zr-DFO-Bn-NCS synthon was successfully employed to radiolabel WBCs and SCs. For WBCs, the radiolabeling efficiency with [⁸⁹Zr] Zr-DFO-Bn-NCS was 22.77±5.02% (n=3). (see FIG. 15). While radiolabeling efficiency for SCs, with [⁸⁹Zr] Zr-DFO-Bn-NCS was 41.83±5.02% (n=3) (see FIG. 16). The radiolabeled WBCs and SCs showed ˜90-95% viability as per trypan blue exclusion cell viability assay. The difference in radiolabeling efficiencies between WBCs and SCs were expected due to the difference in their cell sizes and availability of surface proteins for conjugation and radiolabeling. The average cell size in the cell population was measured by TC10 cell counter (Biorad Laboratories, Inc., Hercules, CA, USA) and found to be 4-10 μm for WBCs and 12-20 μm for SCs.

Micro PET Imaging and Biodistribution of ⁸⁹Zr labeled WBCs

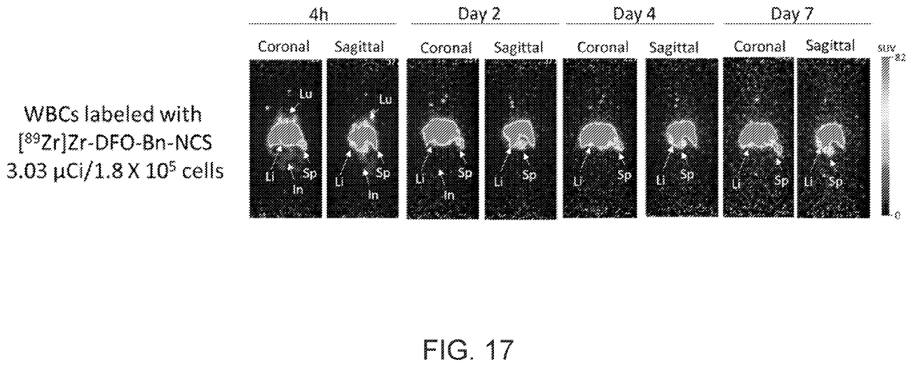

The Micro PET imaging and biodistribution of WBCs were performed independently after radiolabeling of WBCs with [⁸⁹Zr] Zr-DFO-Bn-NCS, to assess any differences in pharmacokinetics of WBCs radiolabeled with different synthons in a healthy athymic mice group (see FIG. 17). After intravenous injection of WBCs-labeled with [⁸⁹Zr] Zr-DFO-Bn-NCS, the majority of the radioactivity was observed in the liver and spleen, and remained significantly high at all time points (4 hours, 2 days, 4 days, 7 days). Importantly, no bone uptake was observed at any time points over 7 days indicating a good in vivo stability of [⁸⁹Zr] Zr-WBCs radiolabeled with [⁸⁹Zr] Zr-DFO-Bn-NCS.

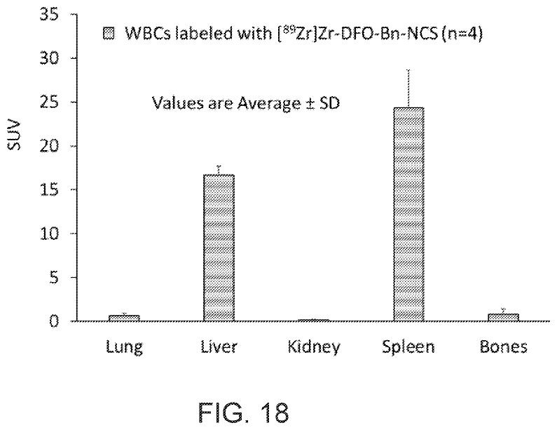

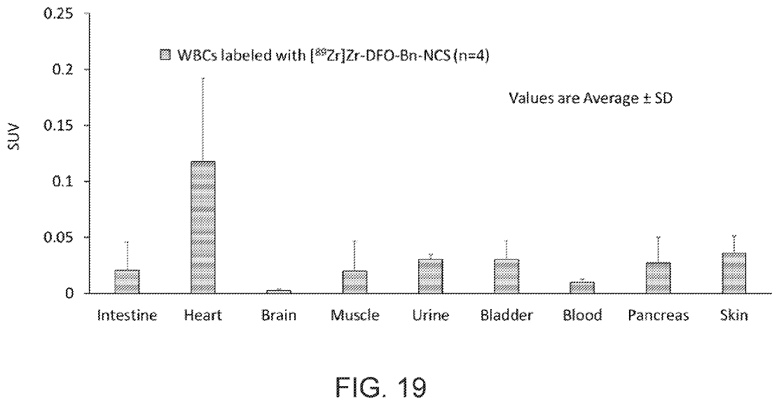

Overall, the in vivo stability of [⁸⁹Zr] Zr-DFO-Bn-NCS demonstrated here was promising]. The biodistribution of radiolabeled WBCs in the rest of the major organs are presented in FIGS. 18-19 and Table 4, indicating mild uptake in lung, heart, muscle, pancreas, and skin at 7 days post injection.

TABLE 4
Uptake (SUV) and biodistribution of white blood
cells (WBCs) labeled with [⁸⁹Zr]Zr-DFO-NCS
(n = 4), in athymic nude mice at day 7 post-injection.

| | White Blood Cells labeled with |
|---|---|
| | [⁸⁹Zr]Zr-DFO-Bn-NCS |
| Organ | SUV Average ± SD |
| Lung | 0.68 ± 0.26 |
| Liver | 16.67 ± 1.05 |
| Spleen | 24.34 ± 4.30 |
| Kidney | 0.19 ± 0.75 |
| Bones | 0.83 ± 0.56 |
| Intestine | 0.02 ± 0.02 |
| Heart | 0.12 ± 0.07 |
| Brain | 0.003 ± 0.001 |
| Muscle | 0.20 ± 0.27 |
| Urine | 0.30 ± 0.0044 |
| Bladder | 0.30 ± 0.017 |
| Blood | 0.0098 ± 0.0030 |
| Pancreas | 0.27 ± 0.23 |
| Skin | 0.36 ± 0.015 |

Micro PET Imaging and Biodistribution of ⁸⁹Zr labeled SCs

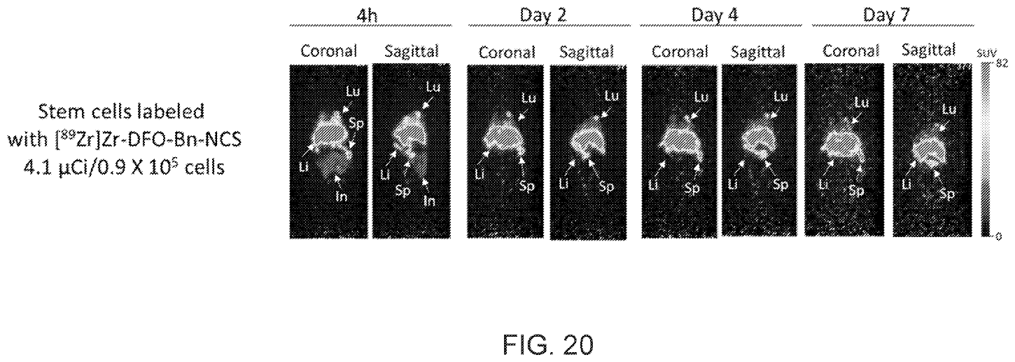

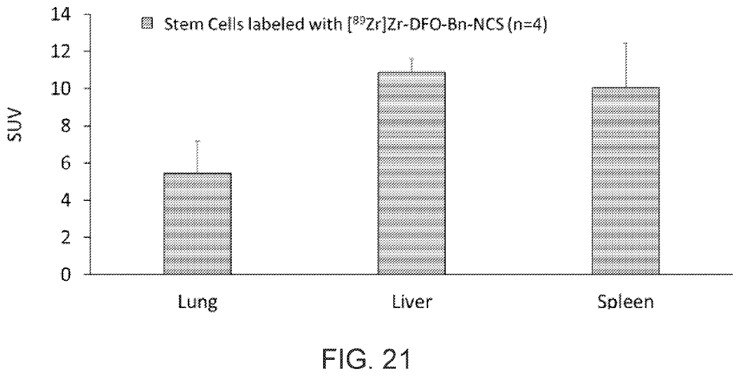

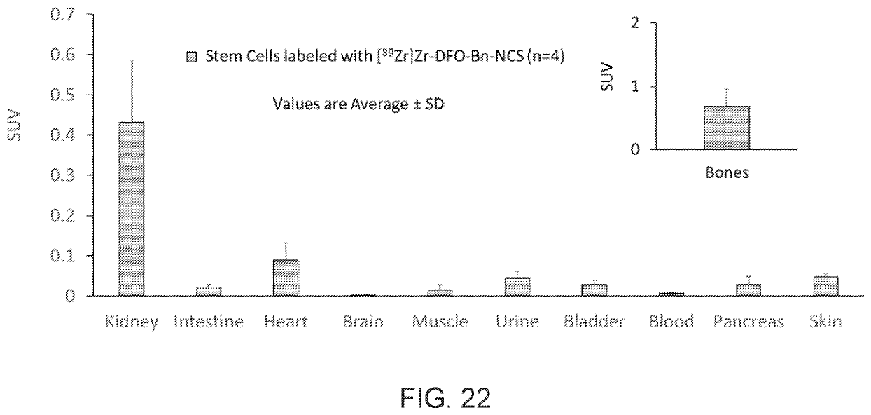

The Micro PET imaging and biodistribution of SCs were performed independently after radiolabeling of SCs with [⁸⁹Zr] Zr-DFO-Bn-NCS, to assess any variation in pharmacokinetics of SCs radiolabeled with different synthons in a healthy athymic mice group (see FIG. 20). The SCs radiolabeled with ⁸⁹Zr using [⁸⁹Zr] Zr-DFO-Bn-NCS showed uptake primarily in lung, liver and spleen at all time points with some early uptake in intestine. The biodistribution of radiolabeled SCs in the rest of the major organs are presented in FIGS. 21-22 and Table 5, indicating mild uptake in lung, heart, kidney, muscle, pancreas, and skin at 7 days post injection.

TABLE 5
Uptake (SUV) and biodistribution of stem cells labeled
with [⁸⁹Zr]Zr-DFO-NCS (n = 4),

| | Stem Cells labeled with |
|---|---|
| | [⁸⁹Zr]Zr-DFO-Bn-NCS |
| Organ | SUV Average ± SD |
| Lung | 5.44 ± 1.72 |
| Liver | 10.88 ± 0.72 |
| Spleen | 10.88 2.37 |
| Kidney | 0.43 ± 0.15 |
| Bone | 0.68 ± 0.27 |
| Intestine | 0.22 ± 0.006 |
| Heart | 0.09 ± 0.04 |
| Brain | 0.002 ± 0.001 |
| Muscle | 0.014 ± 0.013 |
| Urine | 0.44 ± 0.018 |
| Bladder | 0.29 ± 0.01 |
| Blood | 0.0072 ± 0.0025 |
| Pancreas | 0.29 ± 0.20 |
| Skin | 0.47 ± 0.006 |

Micro PET Imaging of Unchelated [⁸⁹Zr] ZrCl₄

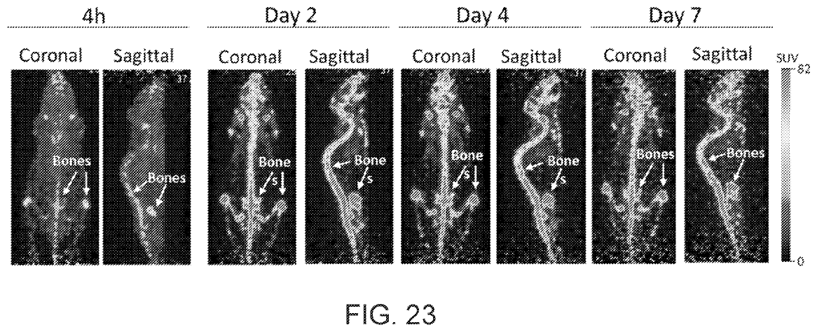

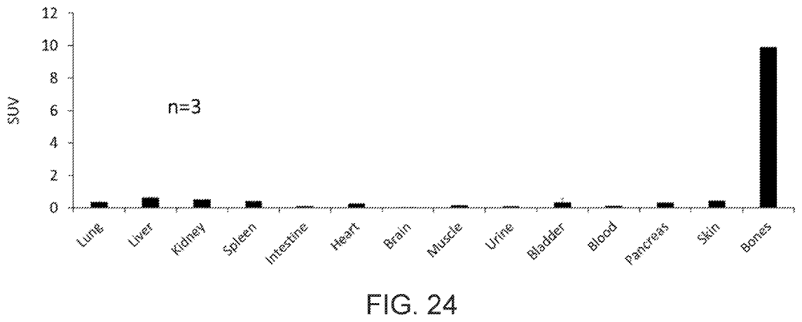

The in vivo characteristics of unchelated [⁸⁹Zr] ZrCl₄ was also investigated (see FIG. 23). The micro PET imaging showed a high accumulation of free ⁸⁹Zr in the bones at 4 hours and did not distribute to the lung, liver, spleen or any other organs at any other time points as it was noticed with radiolabeled WBCS and SCs. The radioactivity increased significantly on day 2 and remain stayed in the bones until day 7, attributing to the entrapment of osteophilic ⁸⁹Zr and its poor clearance from the bones (see FIG. 24 and Table 6).

TABLE 6
Uptake (SUV) and distribution of [⁸⁹Zr]ZrCl4 in athymic
nude mice (n = 3) at day 7 post-injection.

| Free Zr-89 | SUV Average ± SD |
|---|---|
| Lung | 0.36 ± 0.06 |
| Liver | 0.61 ± 0.07 |
| Kidney | 0.49 ± 0.07 |
| Spleen | 0.39 ± 0.09 |
| Intestine | 0.09 ± 0.01 |
| Heart | 0.26 ± 0.03 |
| Brain | 0.02 ± 0.02 |
| Muscle | 0.14 ± 0.05 |
| Urine | 0.08 ± 0.03 |
| Bladder | 0.32 ± 0.09 |
| Blood | 0.12 ± 0.01 |
| Pancreas | 0.29 ± 0.02 |
| Skin | 0.42 ± 0.14 |
| Bone | 9.89 ± 0.4 |

### Conclusion

Both WBCs and SCs were successfully directly radiolabeled with ⁸⁹Zr using ready to use synthons, [Overall, PET based cell radiolabeling methodology offers an effective tool to noninvasively track WBCs, SCs, and other cells to understand the safety, efficacy, distribution and clearance of cell-based therapies.

### LIST OF ABBREVIATIONS USED HEREIN

Bq: Becquerel
DBN: p-isothiocyanato-benzyl-desferrioxamine
DFO-Bz-NCS: 1-(4-isothiocyanatophenyl)-3-[6, 17-dihydroxy-7, 10,18,21-tetraoxo-27-(N-acetylhydroxylamino)-6,11,17, 22-tetraazaheptaeicosine] thiourea
DMSO: Dimethyl sulfoxide
DTPA: Diethylene triamine pentaacetic acid
GBq: Gigabecquerel, 109 Bq
HPLC: High-performance liquid chromatography
IgG: Immunoglobulin G
iTLC: Instant thin layer chromatography
MBq: Mega Becquerel, 10⁶ Bq
PET: Positron Emission Tomography
R_{f}. Retention factor
rTLC: Radioactive-thin layer chromatography
SCs: Stem cells
SD: Standard deviation
SPECT: Single-photon emission computerized tomography
SUV: Standardized uptake value
T_{1/2}: Half-life of radioisotope
WBCs: White blood cells

### REFERENCES

1. Yoon J K, Park B N, Ryu E K, An Y S, Lee S J, Current Perspectives on ⁸⁹Zr-PET Imaging, Int J Mol Sci, 2020 Jun. 17;21 (12): 4309. doi: 10.3390/ijms21124309. PMID: 32560337; PMCID: PMC7352467.
2. Bansal A, Pandey M K, Demirhan Y E, Nesbitt J J, Crespo-Diaz R J, Terzic A, Behfar A, DeGrado T R, Novel ⁸⁹Zr cell labeling approach for PET-based cell trafficking studies, EJNMMI Res, 2015 Mar. 28; 5:19. doi: 10.1186/s13550-015-0098-y. PMID: 25918673; PMCID: PMC4401478.
3. Bansal A, Pandey M K, Yamada S, Goyal R, Schmit N R, Jeon R, Nesbitt J J, Witt T A, Singh R D, Gunderson™, Boroumand S, Li M, Crespo-Diaz R J, Hillestad M L, Terzic A, Behfar A, DeGrado T R, [⁸⁹Zr] Zr-DBN labeled cardiopoietic stem cells proficient for heart failure, Nucl Med Biol, 2020 November-Dec;90-91:23-30. doi: 10.1016/j.nucmedbio.2020.09.001. Epub 2020 Sep. 9. PMID: 32957056; PMCID: PMC7736260.
4. Dasgupta D, Nakao Y, Mauer A S, Thompson J M, Sehrawat T S, Liao C Y, Krishnan A, Lucien F, Guo Q, Liu M, Xue F, Fukushima M, Katsumi T, Bansal A, Pandey M K, Maiers J L, DeGrado T, Ibrahim S H, Revzin A, Pavelko K D, Barry M A, Kaufman R J, Malhi H, IRE1A Stimulates Hepatocyte-Derived Extracellular Vesicles That Promote Inflammation in Mice With Steatohepatitis, Gastroenterology, 2020 Oct; 159 (4): 1487-1503.e17. doi: 10.1053/j.gastro.2020.06.31. Epub 2020 Jun. 20. PMID: 32574624; PMCID: PMC7666601.
5. Bhatt N B, Pandya D N, Wadas T J, Recent Advances in Zirconium-89 Chelator Development, Molecules, 2018 Mar. 12;23 (3): 638. doi: 10.3390/molecules23030638. PMID: 29534538; PMCID: PMC6017441.
6. Vosjan M J, Perk L R, Visser G W, Budde M, Jurek P, Kiefer G E, van Dongen GA, Conjugation and radiolabeling of monoclonal antibodies with zirconium-89 for PET imaging using the bifunctional chelate p-isothiocyanatobenzyl-desferrioxamine, Nat Protoc, 2010 April;5 (4): 739-43. doi: 10.1038/nprot.2010.13. Epub 2010 Mar. 25. PMID: 20360768.
7. Zeglis B M, Lewis J S, The bioconjugation and radiosynthesis of ⁸⁹Zr-DFO-labeled antibodies, J Vis Exp, 2015 Feb. 12; (96): 52521. doi: 10.3791/52521. PMID: 25741890; PMCID: PMC4354640.
8. Holland J P, Sheh Y, Lewis J S, Standardized methods for the production of high specific-activity zirconium-89, Nucl Med Biol, 2009 October;36 (7): 729-39. doi: 10.1016/j.nucmedbio.2009.05.007. Epub 2009 Jul. 29. PMID: 19720285; PMCID: PMC2827875
9. Pandey M K, Engelbrecht H P, Byrne J P, Packard A B, DeGrado T R, Production of ⁸⁹Zr via the ⁸⁹Y (p,n) ⁸⁹Zr reaction in aqueous solution: effect of solution composition on in-target chemistry, Nucl Med Biol, 2014 April; 41: (4) 309-16. PMID: 24607433 DOI: 10.1016/j.nucmedbio.2014.01.006
10. Pandey M K, Bansal A, Engelbrecht H P, Byrne J F, Packard A B, DeGrado T R, Improved production and processing of ⁸⁹Zr using a solution target, Nucl Med Biol, 2016 January; 43: (1) 97-100. PMID: 26471714 DOI: 10.1016/j.nucmedbio.2015.09.007
11. Kasbollah A, Eu P, Cowell S, Deb P, Review on production of ⁸⁹Zr in a medical cyclotron for PET radiopharmaceuticals, J Nucl Med Technol. 2013 March;41 (1): 35-41. doi: 10.2967/jnmt. 112.111377. Epub 2013 Jan. 17. PMID: 23327781.
12. Queern S L, Aweda T A, Massicano A V F, Clanton N A, El Sayed R, Sader J A, Zyuzin A, Lapi S E, Production of Zr-89 using sputtered yttrium coin targets ⁸⁹Zr using sputtered yttrium coin targets, Nucl Med Biol, 2017 July;50:11-16. doi: 10.1016/j.nucmedbio.2017.03.004. Epub 2017 Mar. 23. PMID: 28376350.
13. Pandey M K, DeGrado T R, Cyclotron Production of PET Radiometals in Liquid Targets: Aspects and Prospects, Curr Radiopharm, 2020 Aug. 20 [Epub ahead of print] PMID: 32867656 DOI: 10.2174/1874471013999200820165734
14. Larenkov A, Bubenschikov V, Makichyan A, Zhukova M, Krasnoperova A, Kodina G, Preparation of Zirconium-89 Solutions for Radiopharmaceutical Purposes: Interrelation Between Formulation, Radiochemical Purity, Stability and Biodistribution, Molecules, 2019 Apr. 18;24 (8): 1534. doi: 10.3390/molecules24081534. PMID: 31003494; PMCID: PMC6514948.
15. Pandey M K, Jacobson M S, Groth E K, Tran N G, Lowe V J, DeGrado T R, Radiation induced oxidation of [18F] fluorothia fatty acids under cGMP manufacturing conditions, Nucl Med Biol, 2020 January-Feb;80-81:13-23. doi: 10.1016/j.nucmedbio.2019.11.004. Epub 2019 Nov. 9. PMID: 31759313; PMCID: PMC7056516.
16. Noh, Y. W., Lim, Y. T. & Chung, B. H. Noninvasive imaging of dendritic cell migration into lymph nodes using near-infrared fluorescent semiconductor nanocrystals. FASEB J. 22, 3908-3918 (2008).
17. Yukawa, H. & Baba, Y. In Vivo Fluorescence Imaging and the Diagnosis of Stem Cells Using Quantum Dots for Regenerative Medicine. Anal. Chem. 89, 2671-2681 (2017).
18. Ren, P. G., Lee, S. W., Biswal, S. & Goodman, S. B. Systemic trafficking of macrophages induced by bone cement particles in nude mice. Biomaterials. 29, 4760-4765 (2008).
19. Han, D. & Wu, J. C. Using Bioengineered Bioluminescence to Track Stem Cell Transplantation In Vivo. Methods Mol Biol. 2126, 1-11 (2020).
20. Liu, W.-W. & Li, P.-C. Photoacoustic imaging of cells in a three-dimensional microenvironment. J Biomed Sci. 27, 3 (2020).
21. James, S., Neuhaus, K., Murphy, M. & Leahy, M. Contrast agents for photoacoustic imaging: a review of stem cell tracking. Stem Cell Res Ther. 12, 511 (2021).
22. Kim, T., Lemaster, J. E., Chen, F., Li, J. & Jokerst, J. V. Photoacoustic Imaging of Human Mesenchymal Stem Cells Labeled with Prussian Blue-Poly (I-lysine) Nanocomplexes. ACS Nano. 11, 9022-9032 (2017).
23. Ahrens, E. T. & Bulte, J. W. M. Tracking immune cells in vivo using magnetic resonance imaging. Nat Rev Immunol. 13, 755-763 (2013).
24. Valable S. et al. In vivo MRI tracking of exogenous monocytes/macrophages targeting brain tumors in a rat model of glioma. Neuroimage. 40, 973-983 (2008).
25. Neuwelt, A. et al. Iron-based superparamagnetic nanoparticle contrast agents for MRI of infection and inflammation. AJR Am J Roentgenol. 204, W302-313 (2015).
26. Huang, J., Bao, H., Li, X. & Zhang, Z. In vivo CT imaging tracking of stem cells labeled with Au nanoparticles. View. 3, 20200119 (2022).
27. Yu, C. et al. Enhanced and long-term CT imaging tracking of transplanted stem cells labeled with temperature-responsive gold nanoparticles. J Mater Chem B. 9, 2854-2865 (2021).
28. Kim, T. et al. In Vivo Micro-CT Imaging of Human Mesenchymal Stem Cells Labeled with Gold-Poly-L-Lysine Nanocomplexes. Adv Funct Mater. 27 (2017).
29. Nose, N. et al. [18F] FDG-labelled stem cell PET imaging in different route of administrations and multiple animal species. Sci Rep. 11, 10896 (2021).
30. Wei, W., Jiang, D., Ehlerding, E. B., Luo, Q. & Cai, W. Noninvasive PET Imaging of T cells. Trends Cancer. 4, 359-373 (2018).
31. McCracken, M. N., Tavare, R., Witte, O. N. & Wu, A. M. Advances in PET Detection of the Antitumor T Cell Response. Adv Immunol. 131, 187-231 (2016).
32. Zhang, Y. et al. Tracking stem cell therapy in the myocardium: applications of positron emission tomography. Curr Pharm Des. 14, 3835-3853 (2008).
33. Fu, Y. et al. PET-MRI tracking of imaging-visible microencapsulated stem cells in immunocompetent rabbits. Journal of Cardiovascular Magnetic Resonance. 15, M1 (2013).
34. Galli, F. et al. In Vivo Imaging of Natural Killer Cell Trafficking in Tumors. J Nucl Med. 56, 1575-1580 (2015).
35. Cheng, S.-H. et al. Dynamic In Vivo SPECT Imaging of Neural Stem Cells Functionalized with Radiolabeled Nanoparticles for Tracking of Glioblastoma. J Nucl Med 57, 279-284 (2016).
36. Yang, X. et al. Cellular and molecular imaging for stem cell tracking in neurological diseases. Stroke Vasc Neurol. 6, 121-127 (2021).
37. Zheng, Y. et al. Stem Cell Tracking Technologies for Neurological Regenerative Medicine Purposes. Stem Cells Int. 2017, 2934149-2934149 (2017).
38. Lechermann, L. M., Lau, D., Attili, B., Aloj, L. & Gallagher, F. A. In Vivo Cell Tracking Using PET: Opportunities and Challenges for Clinical Translation in Oncology. Cancers (Basel). 13, 4042 (2021).
39. Stanton, S. E. et al. Concurrent SPECT/PET-CT imaging as a method for tracking adoptively transferred T-cells in vivo. J Immunother Cancer. 4, 27, (2016).
40. Manley, N. C. & Steinberg, G. K. Tracking stem cells for cellular therapy in stroke. Curr Pharm Des. 18, 3685-3693 (2012).
41. Lee, S. B. et al. Tracking dendritic cell migration into lymph nodes by using a novel PET probe 18F-tetrafluoroborate for sodium/iodide symporter. EJNMMI research. 7, 32-32 (2017).
42. Socan, A., Sever, M., Vrtovec, B. & Lezaic, L. Evaluation of CD34+HPCs labeled with ^{99m}Tc-HMPAO. J Nucl Med. 54, 1665-1665 (2013).
43. Peters, A. M. et al. Clinical experience with ^{99m}Tc-hexamethylpropylene-amineoxime for labelling leucocytes and imaging inflammation. Lancet. 2, 946-949 (1986).
44. Dudhia, J. et al. In Vivo Imaging and Tracking of Technetium-99m Labeled Bone Marrow Mesenchymal Stem Cells in Equine Tendinopathy. J Vis Exp. e52748-e52748 (2015).
45. Thakur, M. L. et al. Indium-111-labeled cellular blood components: mechanism of labeling and intracellular location in human neutrophils. J Nucl Med. 18, 1022-1026 (1977).
46. Gholamrezanezhad, A. et al. In vivo tracking of 111 In-oxine labeled mesenchymal stem cells following infusion in patients with advanced cirrhosis. Nucl Med Biol. 38, 961-967, (2011).
47. Ullman, H., Viragh, K., Thomas, M. & Ni, C. 111 In-Labeled White Blood Cell Uptake in the Urinary Bladder in Occult Urinary Tract Infection. Clin Nucl Med. 46, 159-160 (2021).
48. Hughes, D. K. Nuclear medicine and infection detection: the relative effectiveness of imaging with 111 In-oxine-, ^{99m}Tc-HMPAO-, and ^{99m}Tc-stannous fluoride colloid-labeled leukocytes and with 67Ga-citrate. J Nucl Med Technol. 31, 196-201; quiz 203-194 (2003).
49. Jung, K. O. et al. Whole-body tracking of single cells via positron emission tomography. Nat Biomed Eng. 4, 835-844 (2020).
50. Stojanov, K. et al. [18F] FDG labeling of neural stem cells for in vivo cell tracking with positron emission tomography: inhibition of tracer release by phloretin. Mol Imaging. 11, 1-12 (2012).
51. Adonai, N. et al. Ex vivo cell labeling with 64Cu-pyruvaldehyde-bis(N4-methylthiosemicarbazone) for imaging cell trafficking in mice with positron-emission tomography. Proc Natl Acad Sci USA. 99, 3030-3035, (2002).
52. Welch, M. J. et al. Gallium-68 labeled red cells and platelets: new agents for positron tomography. J Nucl Med. 18, 558-562 (1977).
53. Thompson, S. et al. Automated synthesis of [68Ga] oxine, improved preparation of 68Ga-labeled erythrocytes for blood-pool imaging, and preclinical evaluation in rodents. Medchemcomm. 9, 454-459 (2018).
54. Sato, N. et al. ⁸⁹Zr-Oxine Complex PET Cell Imaging in Monitoring Cell-based Therapies. Radiology 275, 490-500 (2015).
55. Man, F. et al. In Vivo PET Tracking of ⁸⁹Zr-Labeled Vgamma9Vdelta2 T Cells to Mouse Xenograft Breast Tumors Activated with Liposomal Alendronate. Mol Ther. 27, 219-229, (2019).
56. Nagengast, W. B. et al. In Vivo VEGF Imaging with Radiolabeled Bevacizumab in a Human Ovarian Tumor Xenograft. J Nucl Med. 48, 1313-1319, (2007).
57. Oude Munnink, T. H. et al. ⁸⁹Zr-trastuzumab PET visualises HER2 downregulation by the HSP90 inhibitor NVP-AUY922 in a human tumour xenograft. Eur J Cancer. 46, 678-684 (2010).
58. Jacobson, O. et al. MicroPET Imaging of Integrin avβ3 Expressing Tumors Using ⁸⁹Zr-RGD Peptides. Mol Imaging Biol. 13, 1224-1233 (2011).
59. Evans, M. J. et al. Imaging Tumor Burden in the Brain with ⁸⁹Zr-Transferrin J Nucl Med. 54, 90-95 (2013).
60. Keliher, E. J. et al. ⁸⁹Zr-Labeled Dextran Nanoparticles Allow in Vivo Macrophage Imaging. Bioconjug Chem. 22, 2383-2389 (2011).
61. Wilks, M. et al. In vivo PET imaging of T-cell trafficking by ⁸⁹Zr-radiolabeled nanoparticles. J Nucl Med. 58, 622-622 (2017).
62. Fairclough, M. et al. A new technique for the radiolabelling of mixed leukocytes with zirconium-89 for inflammation imaging with positron emission tomography. J Labelled Comp Radiopharm. 59, 270-276 (2016).
63. Ferris, T. J. et al. Synthesis and characterisation of zirconium complexes for cell tracking with Zr-89 by positron emission tomography. Dalton Trans. 43, 14851-14857 (2014).
64. Charoenphun, P. et al. [⁸⁹Zr] oxinate4 for long-term in vivo cell tracking by positron emission tomography. Eur J Nucl Med Mol Imaging. 42, 278-287 (2015).
65. Asiedu, K. O., Koyasu, S., Szajek, L. P., Choyke, P. L. & Sato, N. Bone Marrow Cell Trafficking Analyzed by ⁸⁹Zr-oxine Positron Emission Tomography in a Murine Transplantation Model. Clin Cancer Res. 23, 2759-2768.Ccr-16-1561 (2017).
66. Asiedu, K. O. et al. Bone marrow cell homing to sites of acute tibial fracture: ⁸⁹Zr-oxine cell labeling with positron emission tomographic imaging in a mouse model. EJNMMI Res. 8, 109 (2018).
67. Weist, M. R. et al. PET of Adoptively Transferred Chimeric Antigen Receptor T Cells with ⁸⁹Zr-Oxine. J Nucl Med. 59, 1531-1537 (2018).
68. Sato, N. et al. In Vivo Tracking of Adoptively Transferred Natural Killer Cells in Rhesus Macaques Using 89Zirconium-Oxine Cell Labeling and PET Imaging. Clin Cancer Res. 26, 2573-2581 (2020).
69. Man, F., Khan, A. A., Carrascal-Minino, A., Blower, P. J. & R, T. M. d. R. A kit formulation for the preparation of [⁸⁹Zr] Zr (oxinate) 4 for PET cell tracking: White blood cell labelling and comparison with [111|n] In (oxinate) 3. Nucl Med Biol. 90-91, 31-40, (2020).
70. Patrick, P. S. et al. Lung delivery of MSCs expressing anti-cancer protein TRAIL visualised with ⁸⁹Zr-oxine PET-CT. Stem Cell Res Ther. 11, 256, (2020).
71. Tracking peripheral immune cell infiltration of the brain in central inflammatory disorders using [Zr-89] Oxinate-4-labeled leukocytes. ClinicalTrials.gov. https://clinicaltrials.gov/ct2/show/NCT03807973. Accessed 29 Apr. 2020 (2020).
72. Friberger, I. et al. Optimisation of the Synthesis and Cell Labelling Conditions for [⁸⁹Zr] Zr-oxine and [⁸⁹Zr] Zr-DFO-NCS: a Direct In Vitro Comparison in Cell Types with Distinct Therapeutic Applications. Mol Imaging Biol. 23, 952-962 (2021).
73. Lee, S. H. et al. Feasibility of real-time in vivo ⁸⁹Zr-DFO-labeled CAR T-cell trafficking using PET imaging. PLOS One. 15, e0223814 (2020).
74. Patra, M. et al. An octadentate bifunctional chelating agent for the development of stable zirconium-89 based molecular imaging probes. Chem Commun (Camb). 50, 11523-11525 (2014).
75. Deri, M. A. et al. p-SCN-Bn-HOPO: A Superior Bifunctional Chelator for 89 Zr ImmunoPET. Bioconjug Chem. 26, 2579-2591 (2015).
76. Pandya, D. N. et al. Polyazamacrocycle Ligands Facilitate ⁸⁹Zr Radiochemistry and Yield ⁸⁹Zr Complexes with Remarkable Stability. Inorg Chem. 59, 17473-17487 (2020).
77. Rudd, S. E., Roselt, P., Cullinane, C., Hicks, R. J. & Donnelly, P. S. A desferrioxamine B squaramide ester for the incorporation of zirconium-89 into antibodies. Chem Commun (Camb). 52, 11889-11892 (2016).
78. Chomet, M. et al. Head-to-head comparison of DFO* and DFO chelators: selection of the best candidate for clinical ⁸⁹Zr-immuno-PET. Eur J Nucl Med Mol Imaging. 48, 694-707 (2021).
79. Klasen, B., Lemcke, D., Mindt, T. L., Gasser, G. & Rösch, F. Development and in vitro evaluation of new bifunctional ⁸⁹Zr-chelators based on the 6-amino-1,4-diazepane scaffold for immuno-PET applications. Nucl Med Biol. 102-103, 12-23 (2021).
80. Pandey, M. K. et al. A new solid target design for the production of ⁸⁹Zr and radiosynthesis of high molar activity [⁸⁹Zr] Zr-DBN. Am J Nucl Med Mol Imaging. 12, 15-24 (2022).
81. Nguyen et al., Stem cell imaging: from bench to bedside. Cell stem cell, Volume 14, Issue 4, 3 Apr. 2014, pages 431-444

The citation of any document or reference is not to be construed as an admission that it is prior art with respect to the present invention.

Thus, the invention provides methods of labeling of a biological material for medical imaging, methods for preparing a labeling agent, and methods for medical imaging of a subject using a biological material labeled with a labeling agent.

Although the invention has been described in considerable detail with reference to certain embodiments, one skilled in the art will appreciate that the present invention can be practiced by other than the described embodiments, which have been presented for purposes of illustration and not of limitation. Therefore, the scope of the appended claims should not be limited to the description of the embodiments contained herein.

What is claimed is:

1. A method for preparing a labeling agent, the method comprising:
(a) providing a compound including a chelating moiety and a conjugation moiety;
(b) contacting the compound with a radionuclide to create a radiolabeled preparation having a first molar activity measured at an end of step (b); and
(c) purifying the radiolabeled preparation to prepare a labeling agent having a second molar activity measured at an end of step (c), wherein the second molar activity is greater than the first molar activity.

2. The method of claim 1 wherein:
the second molar activity is at least two times greater than the first molar activity.

3. The method of claim 1 wherein:
the first molar activity is in a range of 1 to 50 GBq/μmol.

4. The method of claim 1 wherein:
the second molar activity is in a range of 100 to 500 GBq/μmol.

5. The method of claim 1 wherein:
the radionuclide is selected from the group consisting of ¹¹C, ¹³N, ¹⁵O, ¹⁸F, ^{34m}Cl, ³⁸K, ⁴⁵Ti, ⁵¹Mn, ⁵²Mn, ^{52m}Mn, ⁵²Fe, ⁵⁵Co, ⁶⁰Cu, ⁶¹Cu, ⁶²Cu, ⁶⁴Cu, ⁶⁶Ga, ⁶⁸Ga, ⁷¹As, ⁷²As, ⁷⁴As, ⁷⁵Br, ⁷⁶Br, ⁸²Rb, ⁸⁶Y, ⁸⁹Zr, ⁹⁰Nb, ^{94m}Tc, ^{99m}Tc, ^{110m}In, ¹¹¹In, ¹¹⁸Sb, ¹²⁰I, ²⁰³Pb ¹²¹I, ¹²²I, ¹²³I, and ¹²⁴I.

6. The method of claim 1 wherein:
step (b) comprises contacting the compound with a solution of a halide including a radionuclide cation.

7. The method of claim 6 wherein:
the radionuclide cation is ⁸⁹Zr⁺⁴.

8. The method of claim 6 wherein:
the halide is chloride (Cl⁻).

9. The method of claim 8 wherein:
step (b) comprises contacting the compound with ⁸⁹Zr-chloride in a hydrochloride solution.

10. The method of claim 8 wherein:
step (b) comprises contacting the compound with ⁸⁹Zr-chloride in a hydrochloride solution at a pH in a range of 7 to 9.

11. The method of claim 1 wherein:
step (c) comprises purifying the radiolabeled preparation using reverse phase chromatography.

12. The method of claim 11 wherein:
step (c) comprises purifying the radiolabeled preparation using gradient elution.

13. The method of claim 11 wherein:
the gradient elution uses at least two different solvents.

14. The method of claim 13 wherein:
one of the solvents comprises water and trifluoroacetic acid, and another of the solvents comprises acetonitrile and trifluoroacetic acid.

15. The method of claim 1 wherein:
the chelating moiety is a hydroxamic acid group.

16. The method of claim 15 wherein:
the hydroxamic acid group is a desferrioxamine group.

17. The method of claim 1 wherein:
the conjugation moiety includes an isothiocyanate group.

18. The method of claim 1 wherein:
the conjugation moiety includes a benzyl group.

19. The method of claim 1 wherein:
the labeling agent is a ⁸⁹Zr-isothiocyanato-benzyl-desferrioxamine.

20. The method of claim 1 wherein:
the labeling agent has a radiochemical stability greater than 60% measured at 72 hours after step (c).

21. The method of claim 1 further comprising:
(d) adding a stabilizer to the labeling agent.

22. The method of claim 21 wherein:
the stabilizer is ascorbic acid.

23. The method of claim 21 wherein:
the labeling agent has a radiochemical stability greater than 80% measured at 72 hours after step (d).

24. The method of claim 1 wherein:
step (b) comprises creating the radiolabeled preparation at a radiochemical yield of at least 95%.

25. A method of labeling of a biological material for imaging, the method comprising:
contacting a biological material with a labeling agent prepared by the method of claim 1 such that the biological material becomes labeled for imaging,
wherein the biological material is selected from cells, liposomes, DNA aptamers, RNA aptamers, viruses, nanoparticles, microorganisms, antibodies, proteins, peptides, scaffolds, polymers, and nucleic acids.

26. The method of claim 25 wherein:
a radiolabeling yield when contacting the biological material with the labeling agent is at least 5% when the biological material is contacted with the labeling agent at a concentration of the biological material of 0.1 mg/mL.

27. The method of claim 25 wherein:
a radiolabeling yield when contacting the biological material with the labeling agent is at least 15% when the biological material is contacted with the labeling agent at a concentration of the biological material of 0.5 mg/mL.

28. The method of claim 25 wherein:
a radiolabeling yield when contacting the biological material with the labeling agent is at least 30% when the biological material is contacted with the labeling agent at a concentration of the biological material of 1.0 mg/mL.

29. The method of claim 25 wherein:
a first radiolabeling yield when contacting a first amount of the biological material with a first quantity of the labeling agent is greater than a second radiolabeling yield when contacting a second amount of the biological material with a second quantity of the radiolabeled preparation created in step (b), and
the first amount and the second amount are the same, and
the first quantity and the second quantity are the same.

30. The method of claim 25 wherein:
a first radiolabeling yield when contacting a first amount of the biological material with a first quantity of the labeling agent is at least two times greater than a second radiolabeling yield when contacting a second amount of the biological material with a second quantity of the radiolabeled preparation created in step (b), and
the first amount and the second amount are the same, and
the first quantity and the second quantity are the same.

31. The method of claim 25 wherein:
the biological material is selected from antibodies.

32. The method of claim 25 wherein:
the biological material is selected from proteins.

33. The method of claim 25 wherein:
the biological material is selected from cells.

34. The method of claim 25 wherein:
the biological material is selected from viruses.

35. The method of claim 25 wherein:
the biological material is selected from stem cells.

36. The method of claim 25 wherein:
the biological material is selected from white blood cells.

37. A method for in vivo imaging of a subject, the method comprising:
(a) administering to the subject a biological material labeled with a labeling agent prepared by the method of claim 1;
(b) waiting a time sufficient to allow the biological material to accumulate at a tissue site to be imaged; and
(c) imaging the tissues with a non-invasive imaging technique,
wherein the biological material is selected from cells, liposomes, DNA aptamers, RNA aptamers, viruses, nanoparticles, microorganisms, antibodies, proteins, peptides, scaffolds, polymers, and nucleic acids.

38. The method of claim 37 wherein:
the non-invasive imaging technique is selected from positron emission tomography imaging, positron emission tomography with computed tomography imaging, or positron emission tomography with magnetic resonance imaging.

39. A method of imaging a subject by emission tomography, the method comprising:
(a) administering to the subject a biological material labeled with a labeling agent prepared by the method of claim 1;
(b) using a plurality of detectors to detect gamma rays emitted from the subject and to communicate signals corresponding to the detected gamma rays; and
(c) reconstructing from the signals a series of medical images of a region of interest of the subject,
wherein the biological material is selected from cells, liposomes, DNA aptamers, RNA aptamers, viruses, nanoparticles, microorganisms, antibodies, proteins, peptides, scaffolds, polymers, and nucleic acids.

40. An imaging method comprising acquiring an image of a subject to whom a detectable amount of a biological material labeled with a labeling agent prepared by the method of claim 1 has been administered, wherein the biological material is selected from cells, liposomes, DNA aptamers, RNA aptamers, viruses, nanoparticles, microorganisms, antibodies, proteins, peptides, scaffolds, polymers, and nucleic acids.

41. The method of claim 40, further comprising:
acquiring the image using positron emission tomography imaging, positron emission tomography with computed tomography imaging, or positron emission tomography with magnetic resonance imaging.

42. The method of any of claims 37 to 41 wherein:
the biological material is selected from antibodies.

43. The method of any of claims 37 to 41 wherein:
the biological material is selected from proteins.

44. The method of any of claims 37 to 41 wherein:
the biological material is selected from cells.

45. The method of any of claims 37 to 41 wherein:
the biological material is selected from viruses.

46. The method of any of claims 37 to 41 wherein:
the biological material is selected from stem cells.

47. The method of any of claims 37 to 41 wherein:
the biological material is selected from white blood cells.

48. A method for determining radiolabeling efficiency when a biological material is contacted with a labeling agent including a radionuclide to produce a radiolabeled biological material, the method comprising:
separating the radiolabeled biological material produced when the biological material is contacted with the labeling agent from free radionuclide and unconjugated labeling agent using instant thin layer chromatography.

49. The method of claim 48 wherein:
the labeling agent is the labeling agent prepared by the method of claim 1.

50. The method of claim 49 wherein:
wherein the biological material is selected from cells, liposomes, DNA aptamers, RNA aptamers, viruses, nanoparticles, microorganisms, antibodies, proteins, peptides, scaffolds, polymers, and nucleic acids.

51. The method of claim 49 wherein:
wherein the biological material is selected from antibodies.

52. The method of claim 48 wherein:
the instant thin layer chromatography uses an acid-alcohol mixture as a mobile phase and a gel as a solid phase.

53. The method of claim 52 wherein:
the acid-alcohol mixture comprises citric acid and methanol.

54. The method of claim 52 wherein:
the gel comprises silica.
